(19) United States
(12) Patent Application Publication
(10) Pub. No.: US 2014/0170679 A1
(43) Pub. Date: Jun. 19, 2014

(54) METHOD AND SYSTEM FOR CELL DETECTION AND ANALYSIS

(71) Applicants: James Stewart Aitchison, Toronto (CA); Lu Chen, Toronto (CA); James Jiahua Dou, Toronto (CA); Rakesh Kumar Nayyar, Toronto (CA)

(72) Inventors: James Stewart Aitchison, Toronto (CA); Lu Chen, Toronto (CA); James Jiahua Dou, Toronto (CA); Rakesh Kumar Nayyar, Toronto (CA)

(21) Appl. No.: 14/003,215

(22) PCT Filed: Mar. 7, 2012
(86) PCT No.: PCT/CA12/00227

§ 371 Date: Mar. 4, 2014

Related U.S. Application Data
(60) Provisional application No. 61/449,840, filed on Mar. 7, 2011.

(51) Int. Cl. G01N 33/569; G01N 33/58; G01N 21/64

(52) U.S. Cl. CPC G01N 33/56972; G01N 21/6486; G01N 33/582
USPC 435/7.24; 435/288.7; 435/287.2; 435/34

(57) ABSTRACT

The present invention is a method and a system of cell detection and analysis. The present invention may incorporate at least an optical source, a fluidic chip and a detection module. Cells may be caused to flow within the fluidic chip and specifically past a detection window section accessible by the optical source. The flowing cells may be identified and/or analyzed. The detection module may specifically count the cells of interest as they flow past the detection window section of the chip. The detection module may further be operable to generate or otherwise capture images of the cells as they flow past the window and to use these images collectively for the purpose of analyzing the cells. The present invention may be portable and operable in remote locations.

### FIELD OF INVENTION

This invention relates in general to the field of particle detection and analysis and more particularly to particle detection and analysis using a fluidic chip.

### BACKGROUND OF THE INVENTION

The enumeration of microscopic particles like white cells, bacteria and viruses in samples such as human body fluids are of primary importance in determining the state of human health. Examples of clinical importance include counting of CD4 T-cells in HIV positive subjects and of granulocytes/platelets in patients receiving chemotherapy. Currently, flow cytometry is a frequently used tool for rapid blood cell analysis. Flow cytometry is a technique whereby particles are suspended in a stream of fluid and passed through an optical detection region at a high speed. Due to the application of sheath flow and hydrodynamic focusing, particles are arranged in a single file, and each particle may be individually interrogated by a light beam. The detection and analysis of the particles based on the optical properties of target particle population, such as fluorescence and/or scattering signals.

Although flow cytometer is a mature, widely used instrument, conventional flow cytometry remains largely inaccessible for global routine clinical use due to the size and the cost of such systems. The prior art flow cytometer instruments require complex infrastructure and highly trained personnel to operate. These limitations render flow cytometry too expensive and challenging to support in resource poor settings and remote areas.

Many of the prior art particle manipulation, sorting, counting, analyzing and/or selection systems and units involve large, cumbersome analysis systems. Such systems can be difficult to transport and difficult to utilize in certain situations. Moreover, some prior art systems may include internal mechanisms specific to one type of particle, and therefore be unable to analyze other types of particles.

Examples of related prior art systems include the invention disclosed in U.S. Patent Application Publication No. 2006/0024756, which is a means of moving magnetically labeled cells through a chamber or cuvet. The chamber of cuvet is placed between two wedge-shaped magnets that influence the movement of the cells. This invention is disclosed to be compact, rugged, affordable and easy-to-use in remote locations.

Another prior art example is disclosed in PCT Application No. PCT/KR2004/001736 and is a device for counting micro particles. This invention is a device that comprises a chip containing micro particles, and a shifter for shifting the position of the chip. An area of the chip is photographed when the chip is in a specific position, and the shifter causes the chip to be shifted to a position that is a predetermined distance at a predetermined time interval, to cause an area adjacent to the area photographed just before the shift to be in position to be photographed. In this manner the chip is shifted and photographed until all of the sub-areas on the chip are photographed. The photographs are analyzed and the number of micro particles in each sub-area is counted. The number of micro particles counted in each sub-area is added together to calculate the total number of micro particles in the sample.

Yet another prior art example is disclosed in PCT Application No. PCT/EP2006/068153 and is a device and method for detecting particles. This invention involves the displacement of particles located within a reaction chamber. A number of displacers may be included in the invention, and the displacers may be of a variety of types. For example, a displacer may be a means allowing the vertical movement of a first surface and/or a second surface, or at least one or more parts thereof, relative to each other. Displacement of the particles is intended to facilitate detecting/determining of a value indicative of the presence and/or number of one or more species of particles.

Still another prior art example is disclosed in PCT Application No. PCT/EP2009/053106 and is a method for assaying a sample for each of multiple assays. In this invention involves a microfluidic device that includes multiple test zones within a channel therein. The test zones are contacted with a liquid sample, such as whole blood. The channel may include two walls and at least one of the walls is flexible. The microfluidic device is compressed to reduce the distance between the inner surfaces of the chamber walls. The presence of each analyte is determined by optically detecting an interaction at each of multiple test zones for which the distance between the inner surfaces at the corresponding location is reduced. The interaction at each test zone is indicative of the presence in the sample of a target analyte.

U.S. Patent Application Publication No. 2009/0215072 further discloses related prior art that is an analyte detection device and method that is portable. The device includes a sample reservoir in a cartridge. The sample reservoir includes a mixing chamber where a sample, that is collected by a sample collection device, may react with reagents. An actuator may be coupled to the cartridge to drive fluid through the cartridge. A microsieve-based detection region incorporated in the cartridge. Populations of cells may be mechanically captured on the surface of the microsieve. Light from an optical platform may pass onto the detection region and a detector in the optical platform may acquire images of the sample. The images may be processed an analyzed using software, algorithms, and/or neural networks. The invention may involve chemically sensitizing a population of particles to detect an analyte, so that the presence of a specific analyte in a fluid may be detected by binding to the analyte.

### SUMMARY OF THE INVENTION

In one aspect the present invention relates to a cell detection and analysis system characterized in that it comprises: a fluidic chip incorporating a microfluidic channel operable for one or more cells to flow within the microfludic channel; an optical source positioned to be directed to the fluidic chip or a portion of the fluidic chip; and a detection module operable to capture one or more images of the one or more cells flowing within the fluidic chip.

In one embodiment of the present invention the cell detection and analysis system is further characterized in that the fluidic chip incorporates a detection window and the detection module is operable to capture images of the one or more cells flowing within the fluidic chip through the detection window.

In one embodiment of the present invention the cell detection and analysis system is further characterized in that the optical source is a light source that is positioned either above or below the fluidic chip.

In one embodiment of the present invention the cell detection and analysis system is further characterized in that the detection module incorporates a CMOS detector or a CCD detector.

In one embodiment of the present invention the cell detection and analysis system is further characterized in that the detection module incorporates an image analysis program operable to analyze the one or more images captured by the detection module to produce analysis results.

In one embodiment of the present invention the cell detection and analysis system is further characterized in that the image analysis program may produce diagnostic results.

In one embodiment of the present invention the cell detection and analysis system is further characterized in that the system is portable.

In one embodiment of the present invention the cell detection and analysis system is further characterized in that the fluidic chip, optical source and detection module may be incorporated within a single housing.

In one aspect the present invention relates to a method for cell detection and analysis, characterized in that it comprises the following steps: introducing a cell sample of one or more cells to a fluidic chip; flowing the cell sample through a microfluidic channel within the fluidic chip; and operating an optical imaging module to analyze the cell sample flowing within the fluidic chip.

In one embodiment of the present invention the method for cell detection and analysis is characterized in that it comprises the further steps of: the optical imaging module operating a detector to capture one or more images of the cell sample flowing past a detection window section of the fluidic chip; the optical imaging module operating an image analysis program to analyze the one or more images; and the image analysis program generating cell analysis results relating to the cell sample.

In one embodiment of the present invention the method for cell detection and analysis is characterized in that it comprises the further step of the image analysis program generating diagnostic results relating to the cell sample.

In one embodiment of the present invention the method for cell detection and analysis is characterized in that it comprises the further step of the optical imaging module applying one or more calculations and one or more algorithms to analyze the cell sample.

In one embodiment of the present invention the method for cell detection and analysis is characterized in that it comprises the further steps of: creating a portable device that incorporates the fluidic chip and optical imaging module; and a user carrying the portable device to various locations to perform cell detection and analysis.

In one embodiment of the present invention the method for cell detection and analysis is characterized in that it comprises the further step of carrying the portable device to perform cell detection and analysis in one or more of the following: one or more remote locations; one or more developing locations; one or more developed locations.

In one embodiment of the present invention the method for cell detection and analysis is characterized in that it comprises the further step of storing the analysis of the cell sample of the optical imaging system in a storage means.

In one aspect the present invention relates to an apparatus for cell detection and analysis characterized in that it comprises: one or more housings; a fluidic chip incorporating a microfluidic channel that one or more cells of a cell sample flows through within the fluidic chip; an optical imaging system incorporated in one of the one or more housings, said optical imaging system being operable to capture one or more images of the one or more cells flowing within the fluidic chip when the optical imaging system is positioned to be directed to the fluidic chip or a portion of the fluidic chip; and a cell sample analysis means operable to utilize the one or more images to generate a cell sample analysis result.

In one embodiment of the present invention the apparatus for cell detection and analysis is characterized in that it further comprises the fluidic chip having: an inlet through which the cell sample is introduced to the microfluidic channel; an outlet through which the cell sample may be removed from the fluidic chip; and posts positioned within the fluidic chip.

In one embodiment of the present invention the apparatus for cell detection and analysis is characterized in that it further comprises a waste reservoir positioned near the outlet, said waste reservoir being operable to collect the cell sample after the cell sample has flowed through the microfluidic chip.

In one embodiment of the present invention the apparatus for cell detection and analysis is characterized in that it further comprises the fluidic chip, the optical imaging system and the cell sample analysis means being incorporated in the one housing that is a portable, handheld housing.

In one embodiment of the present invention the apparatus for cell detection and analysis is characterized in that it further comprises a handheld device that is connected to the apparatus whereby the cell sample analysis may be presented to a user by the handheld device.

In one embodiment of the present invention the apparatus for cell detection and analysis is characterized in that it further comprises the optical imaging system and cell sample analysis means being operable to apply multi-fluorescence detection.

In one aspect, the present disclosure relates to a cell detection and analysis system comprising: an optical source; a fluidic chip; and a detection module.

In another aspect, the present disclosure relates to a method for cell detection and analysis, comprising the steps of: introducing a cell sample to a fluidic chip; flowing the cell sample within the fluidic chip past a window detection section; operating a detection module to analyze the cell sample flowing past the window section.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

### BRIEF DESCRIPTION OF THE DRAWINGS

The invention will be better understood and objects of the invention will become apparent when consideration is given to the following detailed description thereof. Such description makes reference to the annexed drawings wherein:

FIG. 1 shows a configuration of an optical imaging system of an embodiment of the present invention.

FIG. 2 shows a configuration of an optical imaging system of an embodiment of the present invention.

FIG. 3a shows an image generated by the particle detection system of the present invention at a time of 0 seconds (T=0 s).

FIG. 3b shows an image generated by the particle detection system of the present invention at a time of 1 second (T=1 s).

FIG. 3c shows an image generated by the particle detection system of the present invention at a time of 2 seconds (T=2 s).

FIG. 3d shows an image generated by the particle detection system of the present invention at a time of 3 seconds (T=3 s).

FIG. 3e shows an image generated by the particle detection system of the present invention at a time of 4 seconds (T=4 s).

FIG. 3f shows an image generated by the particle detection system of the present invention at a time of 5 seconds (T=5 s).

FIG. 4a shows particle distribution inside the analysis chamber of the present invention before the analysis chamber is filled, at a time of 0 seconds (T=0 s).

FIG. 4b shows particle distribution inside the analysis chamber of the present invention after the analysis chamber is filled, at a time of 5 seconds (T=5 s).

FIG. 5 shows a set of four images generated by the CCD detector of the present invention.

FIG. 6a shows a microfluidic chip.

FIG. 6b shows a device layout of a cell/particle detection and analysis microchip, and an enlarged view of a cell/particle detection and analysis microchip layout, in particular showing the posts.

FIG. 7a shows a transmission spectrum of the two half-moon shaped filters used in the present invention.

FIG. 7b shows a transmission spectrum of the two half-moon shaped filters used in the present invention.

FIG. 8a shows an example of plotting of fluid flow speed of the cell sample as flow velocity.

FIG. 8b shows an example of plotting of channel filling time of the cell sample as each lap time.

FIG. 9a shows an image captured by the optical imaging system detector at 50 ms exposure, S/B: 3/2.

FIG. 9b shows an image captured by the optical imaging system detector at 25 ms exposure, S/B: 1300/900.

FIG. 9c shows an image captured by the optical imaging system detector at 15 ms exposure, S/B: 750/550.

FIG. 9d shows an image captured by the optical imaging system detector at 10 ms exposure, S/B: 695/500.

FIG. 10 shows a table providing comparison of results of tests performed on a prior art flow cytometer and the present invention.

FIG. 11 shows linearity test results of a prior art flow cytometer system and the present invention.

FIG. 12 shows a two colour fluorescence image captured by an embodiment of the present invention that incorporates two half-moon shaped optical filters placed together side-by-side in the optical imaging system.

FIG. 13 shows an optical imaging system configuration of an embodiment of the present invention wherein the light source is directed to the upper edge of the disposable cartridge.

FIG. 14 shows an optical imaging system configuration of an embodiment of the present invention wherein the light source is directed to the bottom edge of the disposable cartridge.

In the drawings, embodiments of the invention are illustrated by way of example. It is to be expressly understood that the description and drawings are only for the purpose of illustration and as an aid to understanding, and are not intended as a definition of the limits of the invention.

### DETAILED DESCRIPTION OF THE PREFERRED EMBODIMENT

The present invention is a method, device, computer program product, and system for cell detection and analysis. The present invention may incorporate at least an optical source, a fluidic chip and a detection module, as the core elements. Cells or other particles may be caused to flow through the fluidic chip. The fluidic chip may include a detection window section which is accessible by the optical source. As cells of interest flow through the chip past the detection window section these cells may be identified, for example, such as by illumination by the optical source that may cause cells of interest to fluoresce. The detection module may be utilized to analyze cells of interest, for example, such as the fluoresced cells. The detection module may be operable to function so that cells of interest that flow past the detection window section of the chip may be counted or otherwise analyzed. The detection module may be operable to generate or otherwise capture images of the cells as they flow past the detection window. The captured images may be utilized collectively or individually for the purpose of analyzing the cells.

In one embodiment of the present invention the detection window section of the present invention may be the active area of an optical sensor that is incorporated in the detection module.

The present invention may be operable to provide a simpler, more compact, cost effective and portable cell detection and analysis system than is presently offered by the prior art. The present invention may further achieve better or comparable clinical performance to the known prior art. Due to the compact size of embodiments of the present invention, as well as the fact that in some embodiments its components may be off-the-shelf components, it may have low power requirements, and it may be cost effective to manufacture and utilize, the present invention may be utilized in remote areas as well as developed areas. Therefore, the present invention may be a means of providing on-site cell detection and analysis to areas of the globe where such services are not presently provided because the prior art systems are too large, too expensive, to difficult to source parts for if a part breaks, and cannot be utilized in regions with intermittent power or that lack power, such as remote areas and developing areas. Therefore, the present invention may be operable in remote and developing areas of the world, as well as in developed areas.

In this document the terms “cell” or “cells” may be understood to include all types of particles and particulate matter. The term “cell sample” or similar language references the sample of cells that is provided for use by the present invention and is analyzed and/or counted by the present invention. The term “image” may reference an optical image, or may include other types of images.

One or more of the core elements of the present invention may be contained within a housing, that may be formed of any suitable housing material, such as a plastic material. The size and shape of the housing may vary in accordance with the configuration of the present invention, as described herein. The present invention may be portable and may even be sized so as to be possibly hand held in some embodiments.

In embodiments of the present invention some core elements of the system or device may be external to the housing but linked to the elements within the housing (e.g., by wires, wirelessly, etc.), and in other embodiments other additional elements may be included in the housing along with one or more core elements. The additional elements may enhance the function of the present invention, or may be elements to aid the user of the present invention, such as an emergency signal or other elements to be used by the user of the present invention in specific situations where the present invention may be used, such as in remote areas.

FIGS. 18 and 19 show an example of an embodiment of the present invention that may incorporate some components that are internal to a housing, and other components that are external to the housing. As shown in FIG. 19, the present invention may comprise a portal device 88 that may be operable as a hand held device and is enclosed in a housing. The device may incorporate a display 90 and a keypad 92. Elements required to cause the imaging analysis method and program of the present invention to be operable, and a storage means, may further be incorporated within the housing of the device or these components may be external to the housing. Additionally, an optical imaging system 94 may be incorporated in the device and may extend from the base of the device. The optical imaging system 94 may be positioned above a cartridge 98 that is external to the housing, the cartridge may be a disposable cartridge and may further be a microfludic chip. A light source 96 may be external to the housing and may be positioned to provide light to the upper side of the chip. In this embodiment some components of the invention are housed within a single housing, while other components of the invention are located externally to the housing.

In another embodiment of the present invention, as shown in FIG. 15, components of the present invention may be housed within a single housing 100. An analyzer 102 may incorporate elements required to cause the imaging analysis system and program to be operable, as well as a storage means. An optical imaging system 106, a cartridge 108, that may be a disposable cartridge, and a cartridge housing 110 wherein the cartridge may be positioned, may further be incorporated in the housing.

In yet another embodiment of the present invention, as shown in FIG. 16, the present invention may comprise an analysis device 112 incorporating two sections, namely a device 114 that may be an hand held device, and may be an off-the-shelf device (meaning it may be purchased from a general supplier of parts and is not a custom part designed for this present invention specifically), for example, such as a personal digital assistant, a smart phone, a web book, a portable computer, a laptop computer, a hand held computer, a tablet, or any other portable device. An optical analysis module 116 may be connected to the device. The connection may be a wired connection or a wireless connection. The optical analysis module 116 may function with the device 114 so as to cause the combination of the optical analysis module and the device to provide the present invention. In such an embodiment of the present invention the optical analysis module may perform any of the following activities of capturing images, storing images or other data, and analysis of the images in accordance with the platform of the hand held device. An interface operable to cause the optical analysis module to perform in accordance with the platform of the hand held device, such as a translation interface, may be transmitted or otherwise obtained by either the hand held device or the optical analysis module.

The present invention offers a number of benefits over known prior art methods and systems, including the possible small size of embodiments of the present invention. Some prior art systems involve large cumbersome units, or units that are difficult to utilize in an on-site situation, such as on-site at a remote or developing location, due to the method of use of the unit (e.g., fragile elements are needed for the units, the unit is not portable, the unit cannot be worked except at a specific location, or replacements for any of the unit parts are not readily available if any part should be damaged or break, etc.). One embodiment of the present invention may be configured and designed to be a portable device, such as a hand held device or other portable device, that may be utilized in point of use applications. For example, the portable device may utilized for on-site disease monitoring, water monitoring, or other cell or particulate monitoring, even in remote areas.

The present invention further offers the benefit over the prior art that the present invention does not require repositioning of elements of the invention. Prior art inventions may involve either repositioning of a chip or other sample container, or may require repositioning of a means of viewing the sample, such as a window or chamber. Prior art inventions may further include other moving components in order to measure a given amount of sample volume, such as mirrors for laser scanning, or motion stages for CCD sensor positioning, etc. These prior art inventions can cause inaccuracies in the analysis as areas of the sample may be missed, and consequently results, such as a count of cells in a sample, may be miscalculated due to the missed sections of the sample. The present invention involves analysis that occurs during the flow of a fluidic sample through a chip and more specifically past a detection window section of the chip. Thus, elements of the unit are not moved or repositioned, instead the sample itself moves as it flows past a detection window section.

Moreover, in certain disease diagnosis and monitoring, a minimum amount of sample volume must be examined in order to obtain accurate analysis results. Due to physical limitations of prior art optical imaging systems, a large area of samples must be imaged to meet this requirement. The area of sample to be imaged may exceed the size of the optical detector and the prior art generally compensates by incorporating moving optical sources or detectors to cover the entire sample field. This can cause inaccuracies to pervade the results of prior art inventions. The present invention therefore offers a benefit over the prior art because the present invention involves a fluidic cell sample that flows over the detection module so that the detection module remains in the same position within the invention during the function of the present invention.

A related benefit that the present invention may offer over the prior art is that if a filter is utilized in the present invention, as discussed below, then the filter may not need to be repositioned. Prior art inventions that incorporate a filter generally require that a filter analysis of cells occur in a specific area of the device that is distant from the area where other analysis occurs, or that the filter be moved or repositioned within the device. An embodiment of the present invention that includes a filter may position the filter in a location and utilize the filter as the cells flow past the filter. There is therefore no need to move the filter. Moreover, several types of filters (such as a series of coloured filters) may be included in the same general area of the system in the present invention, such as in the detection window section.

As an example of yet another benefit that the present invention may offer over the prior art, the image capturing technique of the present invention may be continuous. Prior art inventions may capture images based upon time delay, or at set intervals of time. This method of capturing images of a sample may lead to an incomplete image set. Aspects of the sample may occur during the intervals between images that are not captured. In the present invention, images are taken continuously, as described below. The images may capture the flow of the cells over the detection window section of the chip. As a result, the present invention may produce a set of images that captures all of the cells.

Still another example of a benefit that the present invention may offer over the prior art, the present invention may be scalable to the type of cells to be analyzed by the system. Some prior art inventions are designed to analyze a specific type of cell or particulate. Therefore, the size and configuration of the prior art inventions are developed to accommodate the specific type of cells. The present invention may be utilized to analyze a variety of types of cells. Due to the fact that cells flow past the detection window section of the fluidic chip, in embodiments of the present invention the detection window section of the fluidic chip may be small. The fact that the present invention may be able to function with a small detection window section can allow the present invention to be designed and configured to be a small unit, such as a hand-held unit, or even a smaller size.

Yet another example of a benefit that the present invention may offer over the prior art is the sample size (of cells) that is required for the present invention may be smaller than the sample size required by prior art inventions. Some prior art inventions requires a large smear of particles. However, as the cells in the sample may be analyzed as the cells flow past the detection window section of the present invention, it is not always necessary to utilize a large smear of particles to produce a useful analysis when utilizing the present invention. All of the cells can be captured as they flow past the detection window section of the present invention, this means that the sample size can be smaller for the present invention and that the results can still be accurate and useful.

Another example of a benefit that the present invention may offer over the prior art is that the possibility of clumping of cells is diminished in the present invention. Prior art inventions can be hampered in that cells may clump within the inventions. Clumped cells can diminish the accuracy of the prior art inventions. In particular, the counting of cells can be erroneous if cells clump in the sample. The present invention may avoid clumping cells due to the fact that cells are analyzed as the cells are in motion (e.g., while the cells flow past the detection window section of the present invention). Thus, the flow of the cells in the present invention may increase the likelihood that the results of the present invention cell analysis will be more accurate than results produced by prior art inventions.

A comparison of the present invention to other prior art cell/particle detection systems shows that the present invention offers a benefit over the prior art systems in that the present invention may eliminate any additional moving components found in prior art systems, such as mirrors for laser scanning, or motion stages for CCD sensor positioning. The present invention may measure a given amount of sample volume without such moving components. To perform certain disease diagnosis and monitoring, a minimum amount of sample volume must be examined in order to obtain accurate analysis results. Due to physical limitations of prior art optical imaging systems, a large area of samples must be imaged to meet this requirement. When the area to be imaged exceeds the size of the optical detector, moving optical sources or detectors to cover the entire sample field becomes inevitable. The system and method of the present invention does not require the moving parts that are incorporated in the prior art, as discussed herein. The elimination of the need for the moving parts causes the present invention to be able to be formed to be of a smaller size than prior art systems. The present invention provides a portable cytometer, so that the imaging approach of the present invention combined with the usage of purposely designed microfluidic chips offers a miniaturized optical particle detection system in comparison to the prior art systems. The present invention may be a wide field dynamic imaging platform. The present invention may further be miniaturized and integrated onto a handheld analyzer for point-of-care global health applications.

The present invention may further offer a benefit over the prior art in that drawbacks of the conventional prior art flow cytometry are overcome in the present invention through the incorporation of microfabrication techniques and lab-on-a-chip technologies. The present invention may be a miniaturized mobile cell analysis system that is a microfluidic lab-on-a-chip device. These aspects of the present invention offer a number of advantages over the prior art, including small sample requirement, fast and rapid analysis process, the ability to incorporate volume-efficient optical and electrical components, and the provision of portable and inexpensive instrumentation.

The present invention offers a fully functional portable microfluidic based cytometer. The sensitivity of the system of the present invention may be at least 10̂4 MESF and may be functional with a wide range of fluorophores to allow for clinically relevant assays to be implemented. The throughput of the instrumentation of the present invention may also be optimized to reduce the analysis wait time and achieve precise fluidic flow control, as is necessary to ensure the accuracy of testing analysis and results produced by the system and device of the present invention. The mechanical system of the present invention may further be operable to establish multi-colour fluorescence detection assays.

In one embodiment the present invention is an optical platform for cell detection and analysis that forms the basis of a portable cytometer, such as a handheld cytometer. By using a wide field dynamic imaging technique, the present invention may eliminate all moving components that are found in conventional prior art multi-colour fluorescence detection systems. An arrayed filter may be placed in front of the optical detector in the present invention to enable multi-colour fluorescence detection without having any mechanical components.

In one embodiment, the system of the present invention may be comprised of low power, off-the-shelf components (meaning components may be purchased from a general supplier of parts and that are not custom components designed for this present invention specifically). The platform of the present invention may incorporate capillary microfluidic devices, optical systems, as well as an image acquisition and analysis program. The image acquisition and analysis program may incorporate one or more algorithms and other calculations. The image acquisition and analysis program is operable to provide results and reports to the user of the present invention to provide cell analysis for purposes including diagnostic purposes.

One embodiment of the present invention may incorporate a set of calibration beads that demonstrate at least a sensitivity limit of 2000 MESF.

One embodiment of the present invention may be a system, device and method operable to achieve CD4 analysis and counting. Other embodiments of the present invention may be operable to achieve other types of analysis and counting, for example, such as analysis and counting of any of CD3, CD8, CD64, CD4 and CD45 cells. Also embodiments of the present invention may incorporate a system operable to achieve single-colour or multiple-colour functionality, for example, such as two-colour (multiplexed functionality). The present invention may further comprise a system, device and method operable to be used for microscopic particle detection, tracking and counting with sizes in the range from about 1 micron in diameter to about 100 microns in diameter.

An example of four-colour functionality of an embodiment of the present invention is shown in FIG. 21. In this example, a multi-colour fluorescence detection is shown. Specifically a four colour detection incorporating four filters 160, 162, 164 and 166 is applied in FIG. 21. Each filter may be of a different colour and be utilized to identify cells of interest in accordance with each filter. A skilled reader will recognize that embodiments of the present invention may include additional filters and may provide multi-colour fluorescence for more than four colours.

The system of the present invention may include at least the following components in its core: an optical source; a fluidic chip; and a detection module.

The present invention may be powered by a direct connection to a power source, such as an electrical source, or may be integrated with an internal or external battery, which may be a rechargeable battery. The battery may further be a solar powered battery, or a wind-up battery in some embodiments of the present invention.

One embodiment of the present invention may be a system 118, as shown in FIG. 17, that incorporates an analysis device 120 and a cartridge 122. A core system 124 may connect the analysis device and the cartridge. This connection may be achieved through a wired connection or a wireless connection. Elements of the core system may be incorporated in either the analysis device or the cartridge, as shown in FIG. 17.

The analysis device is a portable device, and may be a hand held device. The analysis device may incorporate several elements, for example, such as a display 126, an input/output means 128, CPU 130, a storage or memory means 132, power control 134, and a communication means 136. Elements of the core system that may also be incorporated in the analysis device include elements of an optical imaging system, such as an optical source 138 that may be a light source or other optical source, a lens 140, and a detector 142 that may be a CCD detector or other detector. An image analysis program or system 144 that is part of the core system may further be incorporated in the analysis device. The cartridge may have elements of the core system incorporated thereon, for example, such as a microfluidic device 146 that may be a microfluidic chip or other microfluidic device, and reagents 148 that may be slow-dried chemical reagents or other reagents.

A skilled reader will recognize that the system of the present invention may have other configurations whereby analysis device, cartridge and core system components are incorporated in a single housing, or within a variety of housings, as described herein.

Components of the present invention may be of various types as well. In one embodiment of the present invention the optical source may be a light illuminating source. A free space or fibre/light guide may be coupled with or otherwise connected to the optical source in some embodiments of the present invention. The optical source may also include a free space optical filter and/or a Bragg grating filter that may be integrated in the fiber/light guide. The optical source may further include an optical detector.

In one embodiment of the present invention the fluidic chip may device may be a fluidic device, a microfluidic device, a fluidic cartridge, a microfluidic cartridge, a microfluidic chip, a CCD chip, or some other applicable element. The fluidic chip may incorporate one or more areas, such as a sample loading compartment, a mixing chamber and an analysis chamber. For example, as shown in FIG. 20, in one embodiment of the present invention a fluidic chip may include a sample introduction inlet 150, a sample preparation chamber, a particle analysis chamber and a waste reservoir. The sample may flow along a microfluidic channel 152. The fluidic movement of the cell sample on-chip, and past detector window section 154 in particular, may be completely driven by the capillary force. This may eliminate any need for complex fluidic driving components, such as vacuum pumps. The fluidic chip may further include a detection window section.

The present invention may further incorporate a reservoir 156, as shown in FIG. 20 and an outlet 158.

In embodiments of the present invention the fluidic chip may be disposable. As an example of an application of the present invention utilizing a disposable fluidic chip, the present invention may be utilized as a blood analysis instrument. Such an embodiment of the present invention may include two components: hardware that includes a particle and detection analysis system; and a fluidic chip. A blood sample may be gathered, for example, such as by a pin prick to the skin of a human to draw blood. The blood may be introduced to the fluidic chip, for example, such as by capillary force. The present invention may function as described herein, to undertake analysis and/or detection processes based on the blood sample. When the analysis and/or detection processes are completed the fluidic chip may be disposed of. A skilled reader will recognize that this is just one example of possible uses of the present invention, and in particular of uses of an embodiment of the present invention including a disposable fluidic chip. A skilled reader will further recognize that other uses of the present invention with or without a disposable fluidic chip may be possible.

The fluidic chip of the present invention may be created of a variety of materials, for example, such as glass or polymer substrates. Fluidic flow regulation means to regulate on-chip fluidic flow of the cell sample, including flow past the detector window section, may be included in the fluidic chip, or in the present invention generally. Such fluidic flow regulation means may include, for example, one or more geometric stopping valves, varying microchannel geometries with different fluidic resistances, and/or one or more pumps.

In one embodiment of the present invention the detection module may be an image acquisition and analysis module that may include an optical detector. The optical detector may be of a variety of types, for example, such as a charge coupled device (CCD) image sensor, or a complementary metal oxide semi-conductor (CMOS) image sensor. In some embodiments of the present invention a free space may be integrated with the optical detector. In one embodiment of the present invention, the image acquisition and analysis module may include for example, a 3 mm×0.5 mm rectangular CCD sensor, a CCD sensor with an active detection area of approximately 10.2 mm×8.3 mm, or other applicable sensor. The embodiment may further include driving electric circuits associated with the sensor. A further image analysis program may included in the invention and used to analyze and process the acquired optical images for particle and cell detection and enumeration. A skilled reader will recognize the variety of embodiments of the image acquisition and analysis modules that are possible, as well as the variety of embodiments of the present invention generally that are possible.

The detection module may include a free space optical filter. One or more integrated detectors may further be included in the detection module. The one or more integrated detectors may include a sensor aspect for example, such as an optical active sensor surface that is coated with one or more filters and the coating may be a direct coating, or an independent optical element that may be coated with one or more filters that may be positioned in front of the window section of a fluidic chip. The coating may occur during the manufacturing process or at a later point in time. The integrated detectors may be incorporated with or in the detector window section. The detection module sensor active area may further be divided up into a number of smaller sub-region, and each sub-region may be coated with an optical filter, as described in more detail below.

In one embodiment of the present invention the following method may be applied to utilize the system. For example, in an embodiment of the present invention being utilized, the optical source may include a light source that is either a laser diode or a light emitting diode (LED) device. The light source may serve as an excitation source and may affect the flow of the cells within the microfludic chip.

In some embodiments of the present invention the cell sample may be mixed with another element, for example, such as fluorescently labeled antibodies. The mixing may occur during a sample preparation step. Mixing may be accomplished manually, in a vial, or through a mixing chamber that may be integrated with the present invention and may be included in a microfluidic chip or device utilized in the present invention. A skilled reader will recognize that a variety of methods may be applied to prepare the cell sample to include a mixture.

For example, upon illumination by the light source, fluorescently labeled cells of interest may emit at a different wavelength. The wavelength of the fluorescently labeled cells of interest may be captured by the detector module.

In one embodiment of the present invention an imaging system may include a magnification lens, for example, such as a custom designed three element telescopic 7× or 10× optical lens, or other lens. The magnification lens may magnify the targeted fluorescently labeled cells of interest and may project these cells onto an optical detector.

The samples of cells that are to be tested may be loaded onto a fluidic chip, for example, such as a disposable plastic microfluidic chip, or other fluidic chip. The fluidic chip may be made from hot embossing or injection moulding techniques to produce a specified optical measurement.

After the sample cells are drawn onto the fluidic chip the sample may be mixed with reagents, for example, such as slow-dried or freeze-dried chemical reagents coated on the surface of the fluidic chip. A passive fluidic mixer may be used to prepare the sample for subsequent optical analysis. For example the mixer may prepare the sample by tagging the cells of interest with fluorescently labelled antibodies. The passive mixing approach may eliminate more cumbersome active devices, thus reducing the overall complexity of the system.

An interrogation line or area, defined by the size of the optical detector, for example, such as a CMOS detector or a CCD detector, may be used to examine and measure the excited fluorescence cells and any fluorescence signal. As part of the cell enumeration is completed dynamically in the present invention, a well behaved, orderly flow pattern of cells is ideal for optimum use of the system. To create this type of flow pattern of cells, in one embodiment of the present invention a fluidic channel may be formed in the fluidic chip. The fluidic channel may include a narrow interrogation region that may be designed to create laminar flow of the cells. For example, the interrogation region may be a channel having a depth that is approximately 15-50 micron deep and a width that is less than 500 microns wide. Producing laminar flow, or flow that is strictly laminar, may improve the acquired image quality and increase the accuracy of the dynamic cell detection.

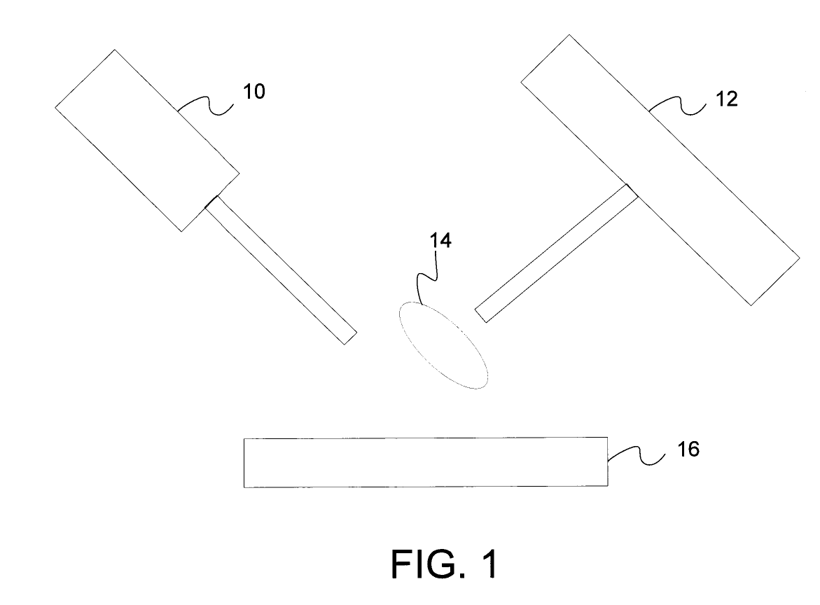

FIG. 1 shows the optical imaging system that may be included in the detection and analysis module of an embodiment of the present invention. In this embodiment the imaging system may utilize fiber optic light delivery and collection components. The use of such fiber optic light delivery 10 and collection components may reduce instrument complexity. Thus use of such light delivery and collection components may further improve robustness and performance of the present invention. In this embodiment the one or more optical detectors 12 may be coated with emission filters. The optic light deliver 10 may further be coated with excitation filter. Any coating on an element of the present invention may be added when the optical detectors are manufactured, or at a later point in time. Coating the one or more optical detectors with emission filters may eliminate additional the need for moving components that are required in prior art fluorescent detection in multicolour or multiplexed analysis.

As further shown in FIG. 1, the present invention may include a lens 14 and disposable cartridge 16 elements.

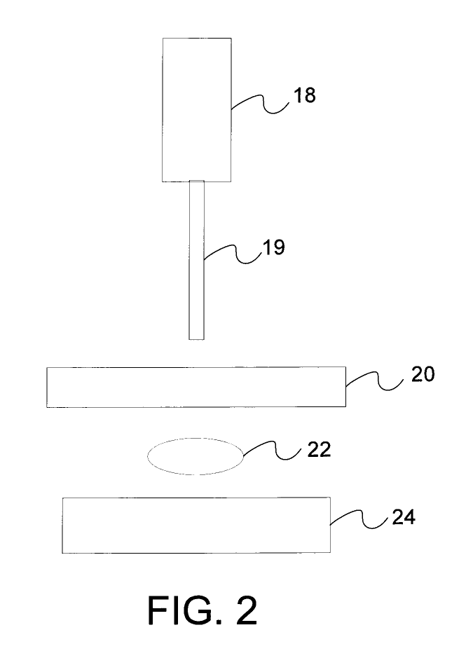

FIG. 2 shows another possible configuration of the optical imaging system that may be included in the detection and analysis module of an embodiment of the present invention. This embodiment of the present invention shown in FIG. 2 may include a light source 18 with a fiber/light guide 19 connected thereto. The fiber/light and/or the light source may be coated with excitation filter. The embodiment may also include a disposable cartridge 20, a lens 22, and a detector 24. The detector may be coated with emission filter. Such an embodiment of the present invention may provide particular benefits over the prior art. For example, this embodiment of the present invention may be more efficient in signal collection since the detector is positioned directly below the sample, which may be a fluorescently labeled sample. A detector may include an imaging lens. The flow and collection of the sample, and the imaging may be primarily dependent on the light collection efficiency of the imaging lens. A skilled reader will recognize that the present invention may be configured in a variety of a ways to achieve the desired results and that FIGS. 1 and 2 are merely examples of possible configurations of the present invention.

The entrance of the cell sample in an analysis chamber of the fluidic chip may trigger the optical detector, such as a CMOS detector or a CCD detector, to being capturing images. The optical detector may capture images of the cells flowing into or through the analysis chamber as the cells pass over or by the detection window section of the fluidic chip. The position of the window and consequently of the optical detector, may vary in accordance with the shape of the fluidic chip.

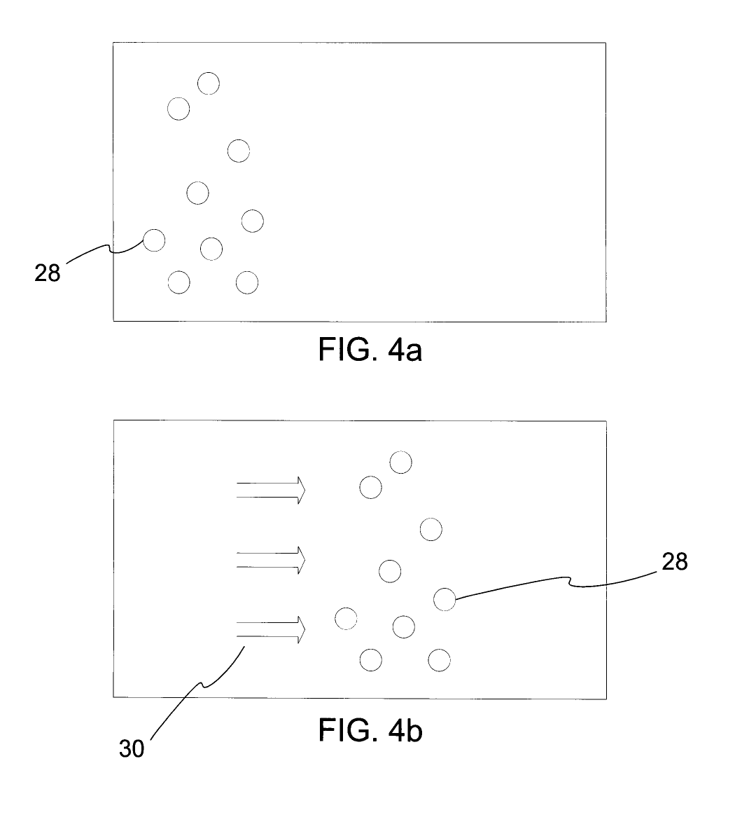

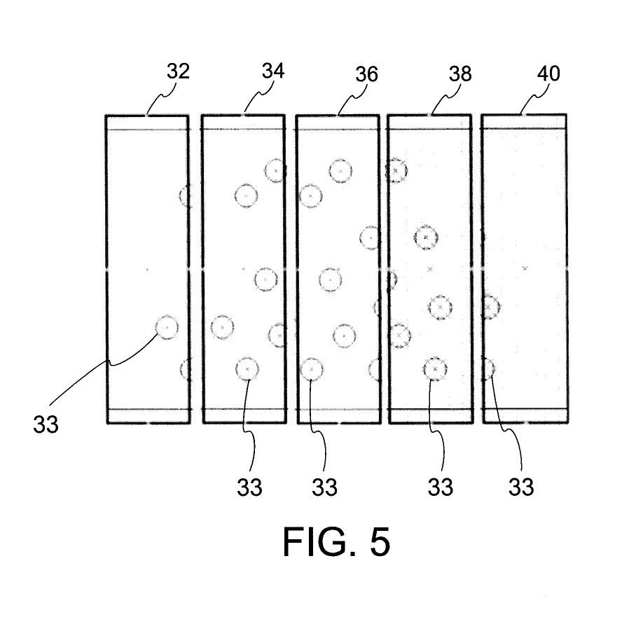

As the fluidic cell sample moves into and fills up the analysis chamber, the optical detector may continue to capture optical images over time. As shown in FIGS. 4a and 4b, the analysis chamber may fill as the cells 28 flow into the analysis chamber. FIG. 4a provides an example of the cells 28 before they being to flow and thereby fill the analysis chamber, at a point in time that is denoted as T=0. FIG. 4b shows the analysis chamber being filled by the cells 28 in a sample at a later point in time (e.g., T=5). The arrows 30 indicate the flow of cells into the analysis chamber that may cause the analysis chamber to become filled, and that in this example the cells have flowed horizontally from the left to the right. A skilled reader will recognize that the flow of the cells may be in a variety of directions in accordance with the size, shape and configuration of the present invention and the fluidic chip specifically.

The analysis chamber may be filled once all of the cell sample has entered the analysis chamber, or before the whole of the cell sample has entered the analysis chamber. The point in time and amount of cell sample that causes the analysis chamber to be filled may depend on the size and shape of the analysis chamber, the type of cells in the cell sample, the amount of cell sample that is gathered, and the minimum amount of sample required to be analyzed in accordance with one or more specific tests to be performed by a user.

Once the entire analysis chamber is completely filled, the image capturing process by the optical detector may stop. Once the optical detector stops capturing optical images all of the captured images may then be combined for analysis. A skilled reader will recognize that embodiments of the present invention may incorporate other points in time or events when images will stop being generated, and that a variety of points in time or events to cause images to stop being generated may be applied in the present invention in accordance with the configuration of the present invention, the nature of the cell sample, etc.

Analysis of the images may involve calculation of the number of cells of interest, for example, such as fluorescent cells, that flow past the detection window section of the fluidic chip. The present invention may generate a total cell count. In one embodiment of the present invention a slow movement of the fluidic cell sample may be maintained inside the fluidic chip. In other embodiments of the present invention a large volume of cell sample may be required to be analyzed. In such embodiments of the present invention a significant number of images may be generated and therefore need to be recorded and processed.

In one embodiment of the present invention, such as a handheld portable system, the captured images may be stored in a memory unit included in the system and contained within the handheld portable system. A graphical processing system may also be included in the portable system, or may be external to the portable system and linked to the portable system. The graphical processing system may be used to process and/or analyze the captured images.

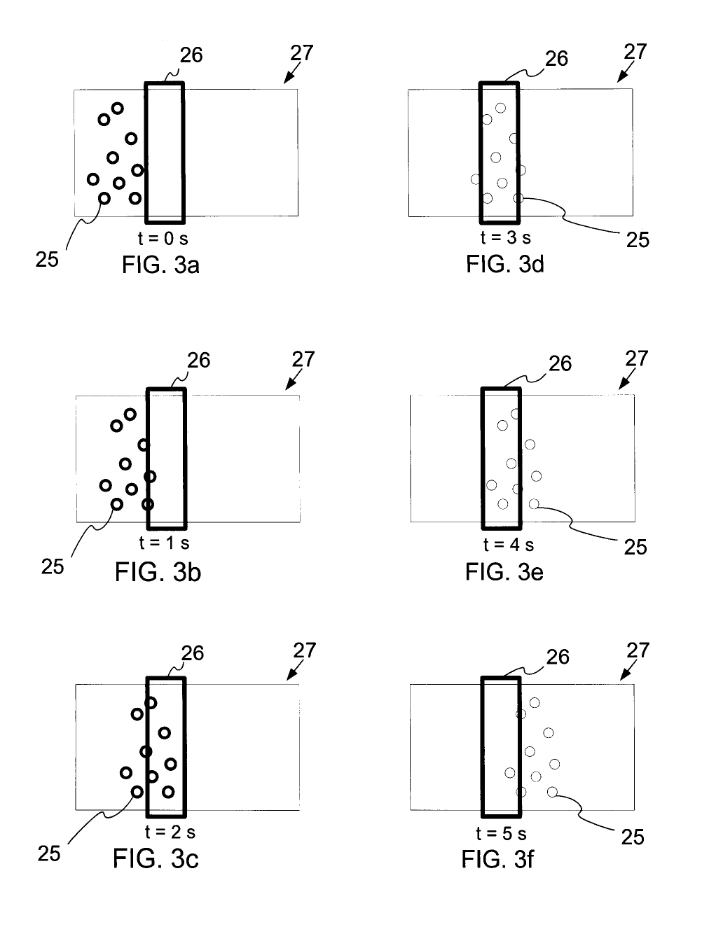

As shown in FIGS. 3a-3f, one or more images may be captured or otherwise generated by the present invention within a time period. The time period may correspond to activities of the present invention, for example, such as the time it takes the analysis chamber to fill with cells, or any other time period for which the present invention is configured to take images. As shown in FIGS. 3a-3f a series of images may be captured or otherwise generated by the present invention. These images may be generated at successive points in time, and the points in time may be regular intervals, or irregular points in time. For example, the images shown in FIGS. 3a-3f are examples of images that are captured by the present invention at different time. T=0 denotes the start of the image capturing acquisition process, and is therefore the point in time when the first image is generated.

FIG. 5 further shows that the images captured by the optical detector of the present invention may be combined together by the analysis module of the present invention. As an example, the captured images 32, 34, 36, 38 and 40 may collectively produce a set of images. The set of images may be analyzed to provide a variety of types of information. The set of images together may show all of the cells 33 passing the detection window section of the fluidic chip. The set of images may be utilized to count the cells in each image to calculate a total number of cells passing by the detection window section. In some embodiments of the present invention redundant images may be captured (as shown in FIG. 5). Capturing redundant images, or redundancy within the images may improve cell detection accuracy in embodiments of the present invention. Embodiments of the present invention may be configured to capture images devoid of redundancy.

Once the analysis chamber of the fluidic chip is filled with the cell sample the detector may then take static optical scattering images of the sample inside the analysis chamber. Such static optical scattering images of the sample may be analyzed by exposure studies to identify finer details of individual cells and of the cell sample generally. Such finer details may provide additional information that is useful to a user of the present invention, for example, such as cell morphology, optical density of the carrier solution, etc. Such information in isolation, or when combined with the other analysis results produced by the present invention based on the cell sample, may be essential in diagnosis and monitoring of diseases and assays, for example, such malaria diagnostics, tuberculosis diagnostics, etc.

The present invention may provide a method whereby optical measurements are performed on sample cells as they flow past the detection window section. The sample cells may be simultaneously propagating inside the fluidic chip. The optical measurements may include fluorescence measurements and scattering measurements.

To achieve fluorescence measurement, multi-colour fluorescence detection steps may be included in the method of the present invention. In particular, the detection module may be utilized to capture images of multi-colour fluorescent dyes, or to otherwise detect such multi-colour fluorescent dyes as the cells of the cell sample pass the detection window section.

An algorithm may be applied to process the images and any other data acquired by the optical detector. For example, motion analysis may be applied to the flowing cells of the cell sample to produce specific data results. As another example, statistical data regarding the cell sample population may be generated from the analysis of the detection module applied to the cell sample generally and the images captured as it flows by the detection window section.

In one embodiment of the present invention, analysis of the cells may occur on the fly, as the cells pass through the detection region. In this embodiment it is not necessary to collect a set of images before beginning an analysis step. Analysis of an image may occur as soon as the image is generated.

In another embodiment of the present invention, a two-phase analysis process may occur. As a first step one or more images may be analyzed upon the generation of the images, whereby each image is analyzed on the fly. As a second step the set of images generated by the present invention during the specified time period (e.g., such as the time required to fill the analysis chamber with cells of a test sample) will be collected and analyzed as a group, as discussed herein.

One embodiment of the present invention may be designed to achieve multiplexed analysis. In such an embodiment multi-colour fluorescence imaging may be achieved. For example, one or more linear detectors may be placed within the detector window section. Each linear detector may be coated with a distinct detection filter. Each linear detector may detect or otherwise highlight one particular colour of fluorochrome of the cells. The particular colour of fluorochrome of the cells to be detected or highlighted may be determined by the specific filter chosen. The detection module may generate images of each of the linear detectors. When two or more independent fluorescence images are superimposed, a multi-colour fluorescence cytometry system may be established. By adding more linear detectors coated with distinct detection optical filters, this system can be expanded into a multi-colour portable and/or handheld cytometer system for a range of analysis and assays.

For example, the detection window section could be coated with panel sections of one or more colours or other filters, such as a red panel, a green panel and a clear section. The incorporation of colours into the detection window section may allow for the testing of several results simultaneously. The coating may be adhered to the top surface of the window section that may be included in the fluidic chip, for example, such as by direct adherence, or the coating may be provided on a transparent optical element that may be positioned in front of the window section.

In embodiments of the present invention the detection window section may incorporate the same colour two or more times. For example, the detection window section may incorporate two panels of red spaced apart from each other. An average of the results collected by the present invention from initial colour panel that the cell sample passes and the panel of the same colour that the cells subsequently passes may be generated by the present invention.

Depending on the configuration of the present invention and the nature of the cell sample, the use of multiple colours or of one or more colours multiple times may verify the accuracy of the present invention and thereby increase the surety of the accuracy of the present invention for some types of analysis. A skilled reader will recognize that such a detection module incorporating one or more colours in the detection window section, and possibly incorporating one or more colours twice in the detection window section, may be utilized for a variety of analysis purposes.

The present invention is a particle detection and analysis system that may comprise a fluorescent excitation and detection platform, as shown in FIG. 2. The platform of the present invention may include a light source 18, for example, such as a laser diode or LED for excitation, emission filters for signal collection and an optical detector 24, for example, such as CMOS or CCD camera for detection. The light source may be connected to an optical fiber/light guide 19 that may be coated with excitation filter. Upon illumination, fluorescently labelled particles of interest may emit at a different wavelength which can then be captured by the optical detector. The imaging system may be an objective lens 22, for example, such as an off-the-shelf microscope objective lens, that is operable to magnify the targeting cells and projects onto the optical detector. The samples to be tested may loaded on a disposable cartridge 20, for example, such as a disposable plastic microfluidic chip. The microfluidic chip may be formed by hot embossing or injection moulding techniques in accordance with applicable optical measurements. The image acquisition and analysis module may comprise a rectangular CCD or CMOS sensor as well as associated driving electric circuits.

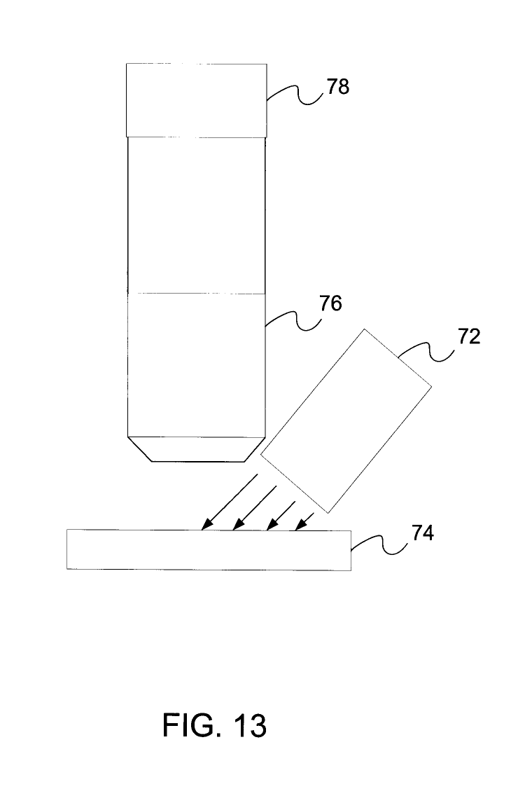

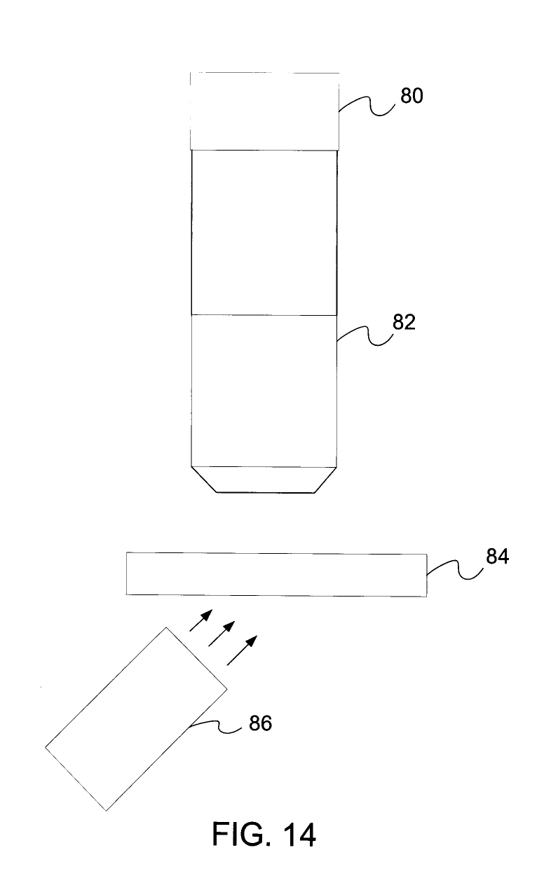

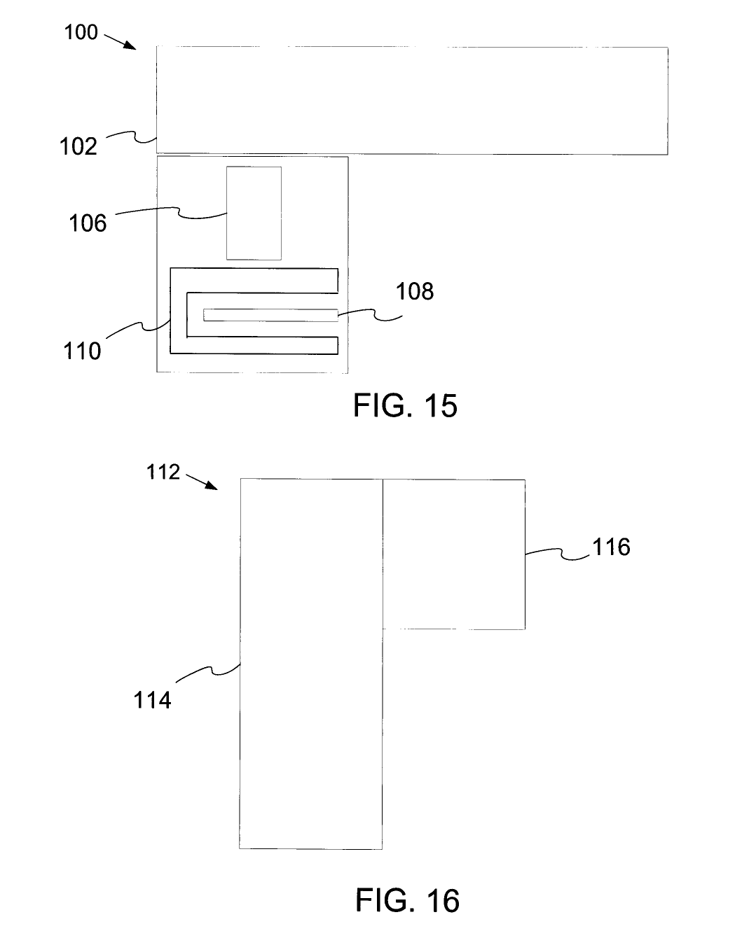

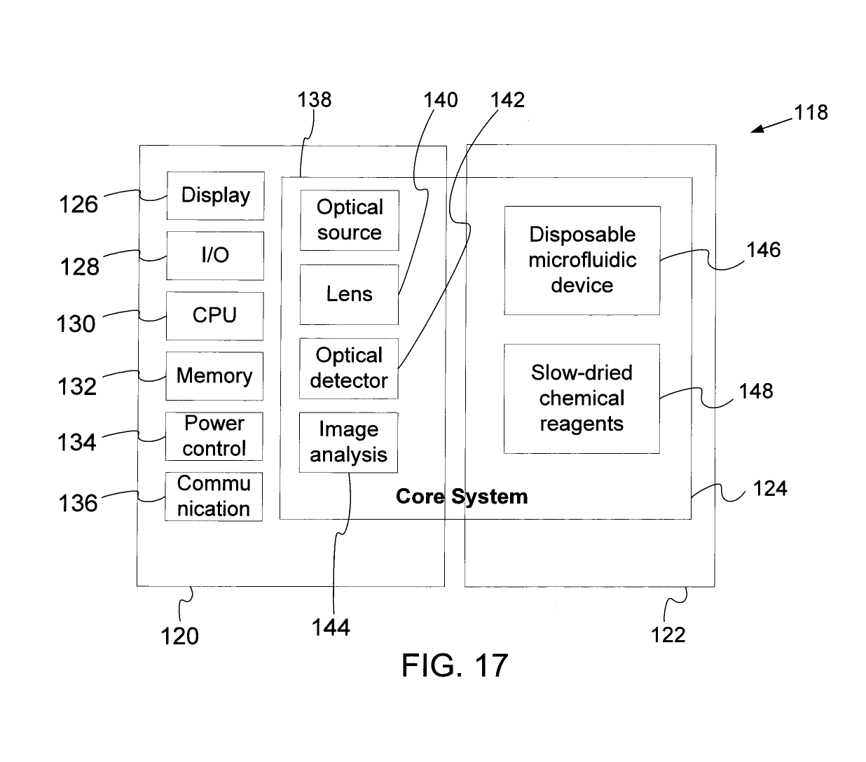

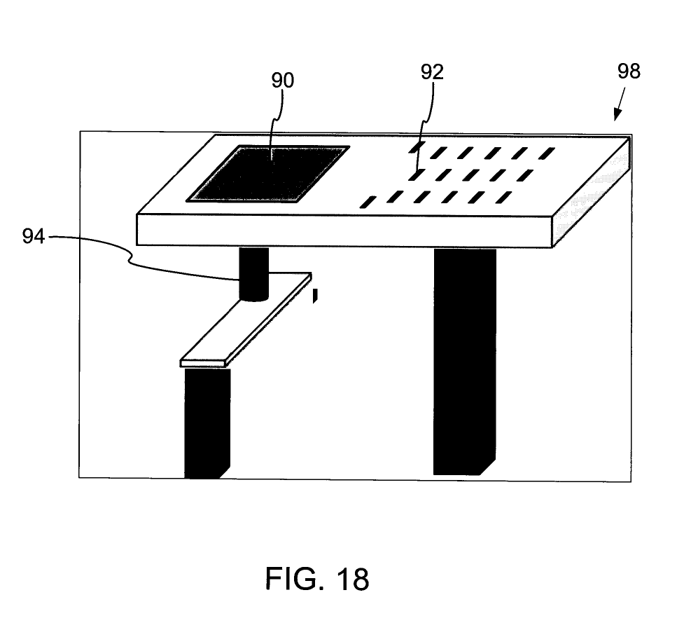

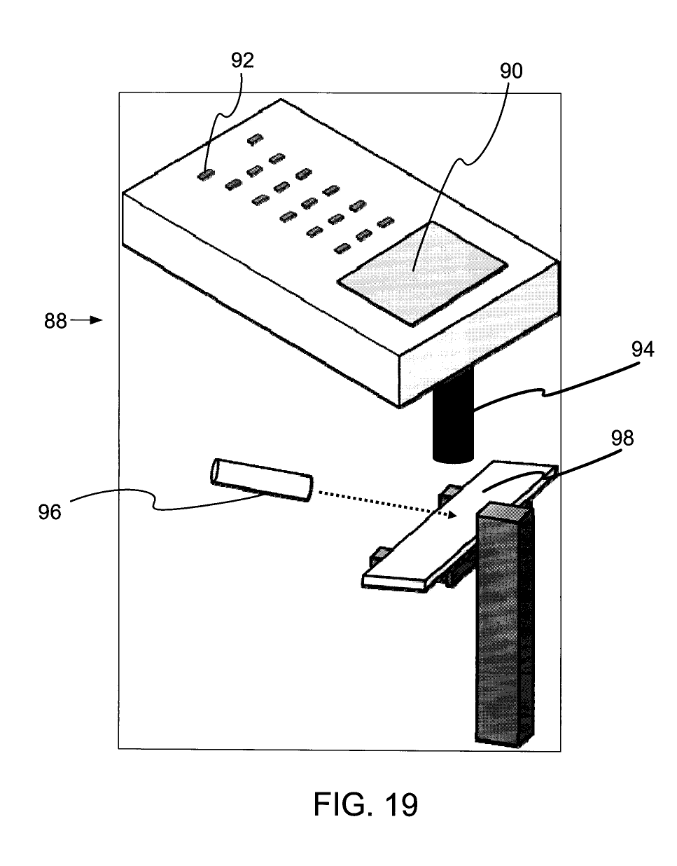

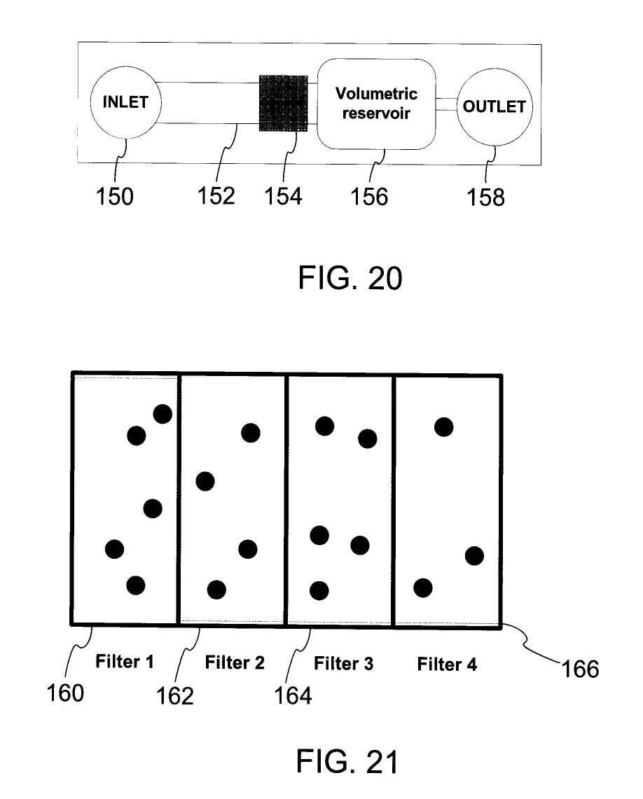

As shown in FIGS. 13 and 14 the configuration of the optical imaging system of the present invention may incorporate the components, including the light source in variant positions. As shown in FIG. 13, in an embodiment of the present invention a detector with emission filter may be connected to an imaging lens 76. The connected detector and lens may be positioned above a disposable cartridge 74. A light source 72 may be positioned above the disposable cartridge and may be angled so as to provide light to the upper surface of the disposable cartridge beneath the imaging lens.

As shown in FIG. 14, in another configuration of the present invention, a detector with an emission filter 80 may be connected to an imaging lens 82. The connected detector and lens may be positioned above a disposable cartridge 84. A light source 86 may be positioned beneath the disposable cartridge and may be angled to provide light to the under-side of the disposable cartridge below the lens. A skilled reader will recognize that other configurations of the elements of the present invention may be possible in embodiments of the present invention.

An image analysis program may be used to analyze and process the acquired optical images for particle and cell detection and enumeration.

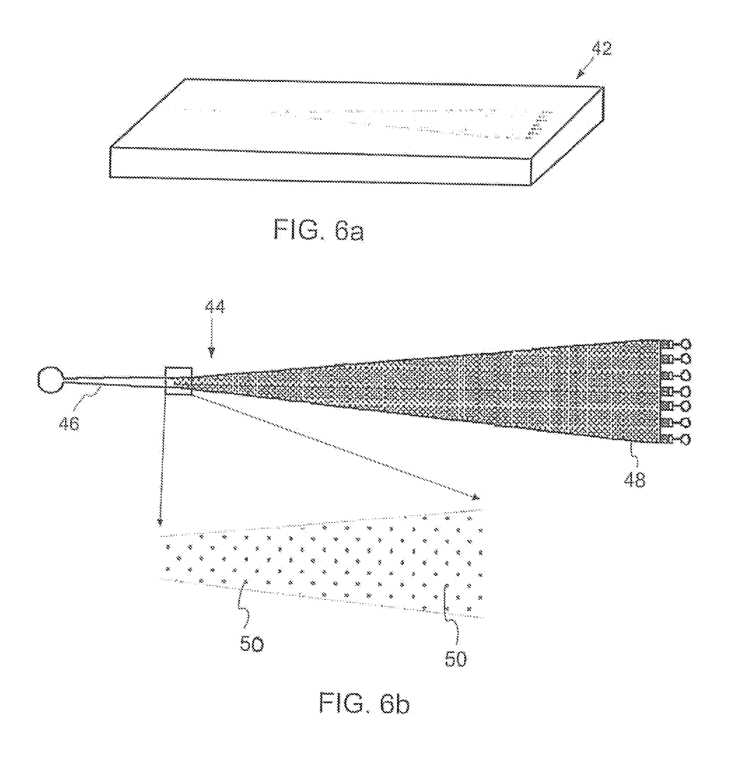

The present invention may incorporate several elements, including elements available off-the shelf. Incorporating off-the shelf elements may decrease the cost of the present invention device and system. For example, a variety of types of beads may be incorporated with the present invention, for example, such as phycoerythrin (PE) (excitation/emission 532 nm/585 nm) and PE-Cy5 (excitation/emission 532 nm/700 nm) labelled beads with 6 μm diameter. As another example, the present invention may incorporate saline, for example, such as 1× phosphate-buffered saline (PBS) (product number BP 2438-4). As still another example, immunotrol, both high and low count controls purchased off-the shelf may be incorporated in the present invention. A microscope objective lens, for example, such as a 10×, NA 0.30, may be incorporated in the present invention. A CCD camera, for example, such as a Pixelfly USB, may also be incorporated in the present invention. The present invention may further incorporate optical filters for fluorescence emission collection, for example, such as 585/40 and 708/75 filters. As yet another example of an off-the shelf optical lens tube assembly may be incorporated in the present invention. A skilled reader will recognize that other components that are not off-the shelf, but are constructed specifically for incorporation in the present invention, or a combination of off-the shelf and components constructed specifically for the present invention, may be utilized with the present invention. The design layout of the present invention and the fabricated polymer microchips 42 incorporated in the present invention are shown in FIGS. 6a and 6b. The microfluidic element may consist of a sample introduction inlet, a sample preparation chamber, particle analysis chamber and waste reservoir. In autonomous microfluidic systems, such as that incorporated in the present invention, on-chip fluidic movement may be purely driven by the capillary force. This eliminates the need for complex fluidic handling and driving components such as vacuum pumps, tubes and sealing in the present invention. An interrogation area, defined by the size of the detector, may be used to examine and measure the excited fluorescence signal. Since the cell enumeration process may be completed dynamically, it may be improve performance of the device and system if a well behaved cells flowing pattern be implemented. The present invention achieves such a well behaved cells flowing pattern, as is described herein.

In the present invention the microfluidic channel 44 of the narrow interrogation region may be designed in such a way that the flow is strictly laminar. For example, the narrow interrogation region 46 may be designed to be an appropriate size to achieve the flow required to allow the present invention to achieve optimum function. The channel of one embodiment of the present invention may be about 15 microns deep and over about 800 microns wide. A skilled reader will recognize that this is an example of a size of a narrow interrogation region that may be incorporated in the present invention and that other sizes of interrogation regions may be incorporated in the present invention.

The design of the narrow interrogation region to achieve specific flow may have the result of improving the acquired image quality and making the dynamic cell detection more accurate. Beyond the detection point, the channel may widen 48 in order to reduce the fluidic resistance and improve flow speed. This unique design may allow the fluid flow in the present invention to maintain uniform speed throughout the analysis time. Once the chamber is filled a known volume of sample may be analyzed. For example, this is can be an important analysis criteria in CD4 T cell enumeration analysis.

The microfluidic channel may include one or more posts 50, as shown in the enlarged section of FIG. 6c. The posts may be of a variety of sizes, for example such as about 100-micron wide. The posts may be positioned at regular, uniform intervals within the channel, or may be more randomly spaced. The posts may be positioned so as to affect the flow of the cells, and may even be positioned to affect the direction of the flow or the flow speed. The posts may further act to keep the cells from clumping. As discussed herein clumping of cells can negatively affect the results of cell analysis, therefore the posts may act to prevent cells from clumping, and may therefore contribute to the increased accuracy of the analytic results of the present invention.

The posts may further be of a height to separate layers of the chip, and therefore may cause the space between the layers to vary. In this manner the posts may be utilized to impede or facilitate the flow of particular cells in accordance with the size of the particular cells. For example, the space between the layers of the chips at particular points may be determined by the height of the posts that support the layers in a position that keeps each layer at a distance from the other layer. In the circumstance that a cell is larger than the space between the layers of the chip at a particular area of the chip the cell may be impeded from flowing in this area of the chip and may be forced to flow in a direction within the chip where the space between the layers of the chip is sufficient to be larger than the size of the cell.

Microfluidic chips to be utilized in the present invention, that may be cartridges, for example, such as disposable cartridges, may be fabricated using a photolithography technique. Wafers of the chips may be packaged using a laminar press process. The base layer of the chip may be made of a variety of materials, for example, such as plastic acrylic. The base layer, or underlayer, may incorporate fluidic structures, for example, such as structures defined in a SU-8 negative photoresist. The microfluidic channels may be patterned using photolithography technique, so that initially an underlayer may be deposited, for example, the depositing step may involve spin coating and drying techniques. The underlayer may be fully cured.

Next, a second layer may be deposited, for example, such as through the same steps as utilized to deposit the underlayer. The second layer may further be patterned, for example, such as by exposing through a photomask. Exposed samples may be baked and developed to form the desired features, as required to function in the present invention.

The lid layer, or capping layer, of the chip may also be made of a variety of materials, for example such as a plastic acrylic. The lid layer may be a partially cured SU-8 photoresist layer deposited with mechanically drilled through holes to form the inlets and outlets.

The base and the capping layers may be assembled in registration in bonding jogs and may be heated under mechanical load in a laminating press. The base and capping layers may be held for a period of time to form the bond.

If disposable cartridges are produced by mass production, the disposable cartridge may be formed by using injection molding or hot embossing process.

The reagents, antibody conjugated with fluorescent dyes, either in liquid or solid powder form, may be stored or preloaded inside the disposable cartridge during fabrication.

The present invention may involve a measurement step. As shown in FIG. 2, the optical imaging system of the cell/particle detection and analysis platform of the present invention may differ from a standard, prior art fluorescence imaging approach in that the present invention may not include either an emission filter or any dichroic mirrors. For multi-colour detection, a custom designed emission filter may be incorporated in the present invention in the optical detection system.

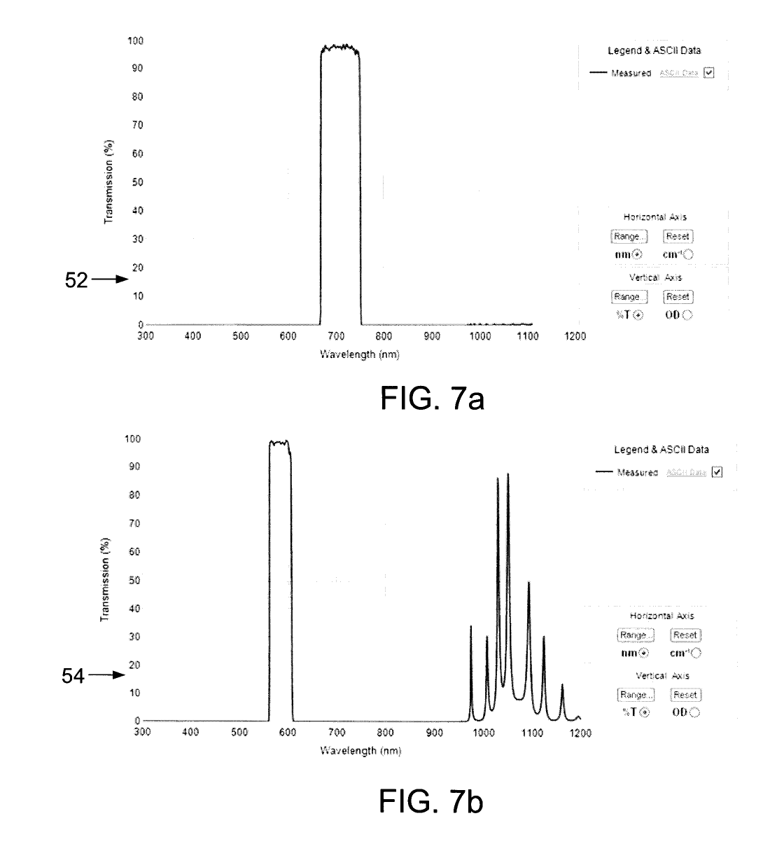

Filter set outputs, as shown in FIGS. 7a and 7b, may be shown as a transmission spectrum of the filter used in the present invention 52 and 54. These spectrums may represent the optical property of the present invention. The spectrum 52 shown in FIG. 7a represents the filter set output of an example of measured ASCII data of one embodiment of the present invention. The spectrum 54 shown in FIG. 7b represents the filter set output of an example of average ASCII data of one embodiment of the present invention. The horizontal axis of the filter set for the embodiment of the present invention relating to both FIGS. 7a and 7b involve a horizontal axis that has a range indicated in nm and a reset to cm⁻⁴, and a vertical axis that has a range indicated in % T and a reset to OD.

The filter set of the present invention may incorporate two half moon shaped fluorescence filters in a single cell. This filter set may allow for two-colour fluorescence detection simultaneously.

The imaging system of the present invention may utilize a wide field dynamic imaging approach. Such an imaging system may eliminate any moving components from being incorporated in the present invention. For example, the present invention may not incorporate any moving components, such as filter wheels and rotation stages. Prior art systems require such moving components, such as filter wheels and rotation stages, in order to achieve fluorescent detection in multi-colour (or multiplexed) analysis. The present invention is operable to achieve fluorescent detection in multi-colour (or multiplexed) analysis without such moving components. This is yet another benefit of the present invention over the prior art.

During analysis performed by the present invention, a time series of particles moving inside the microfluidic device may be captured. An illustration of the particle detection system of the present invention is shown in FIGS. 3a-3f. The six figures, FIGS. 3a-3f, show snapshots of the image acquisition system of the present invention at different points in time during an analysis stage. In this example, T=0 denotes the start of the image acquisition. During the analysis stage, the detector, that may be a CMOS detector or a CCD detector, may continually take images as cells 25 move into an area of a detection window 26 that is located within the area of the microfluidic chip 27.

As examples of the movement of cells during the analysis stage in relation to the detection window at points in time during the analysis stage, FIG. 3a shows an image captured by the detector at T=0, FIG. 3b shows an image captured by the detector at T=1 second, FIG. 3c shows an image captured by the detector at T=2 seconds, FIG. 3d shows an image captured by the detector at T=3 seconds, FIG. 3e shows an image captured by the detector at T=4 seconds, and FIG. 3f shows an image captured by the detector at T=5 seconds.

A skilled reader will recognize that images shown as FIGS. 3a-3f are merely examples of possible images of cell movement in the detection window during an analysis stage of the present invention, other images or cell movement may be produced by other embodiments and various analyses performed by the present invention. Furthermore, a skilled reader will recognize that the time intervals when the detector, that may be a CMOS detector or a CCD detector or other type of detector, may capture images may be any regular or irregular interval. FIGS. 3a-3f show images captured in 1 second intervals, but the detector may capture images at any interval during the analysis stage.

When the present invention is utilized, the entrance of a sample, for example, such as a blood sample, into the analysis chamber may trigger the detector, for example, such as a CMOS detector or a CCD detector, to begin a data acquisition stage or process. The data acquisition performed by the present invention may include acquiring images, as described herein. An analysis of the acquired images may involve a calculation of the number of particles or cells, for example, such as fluorescent particles of interest, that flow by the detection window. This calculation can be utilized by the present invention to generate a final cell count.

In some embodiments of the present invention, due to the slow fluidic movement inside the microfluidic chip and the large volume of the sample to be analyzed, a significant amount of images may need to be recorded and processed.

The present invention may incorporate a microfluidic device that comprises an introduction inlet whereby sample cells may be introduced to the device, a preparation chamber where sample cells may be prepared for analysis, a cell analysis chamber wherein cells may be analyzed as discussed herein, and a waste reservoir for collecting waste cells. The inlets and chambers of the microfluidic device of the present invention are connected so that the introduction inlet is connected to the preparation chamber, so that the cells introduced to the device may flow into the introduction inlet, through the introduction inlet and into and through the preparation chamber. The preparation chamber is connected to the analysis chamber, so that the cells may flow from the preparation chamber into and through the analysis chamber. The waste reservoir is connected to the analysis chamber, and may be connected to the preparation chamber in some embodiments of the present invention, so that cells may flow into the waste reservoir and be collected in the waste reservoir.

The cells collected in the waste reservoir may be disposed of, for example through removal of the waste reservoir from the device and disposal of the cells collected therein. In such an embodiment of the present invention, the waste reservoir may be reattachable to the present invention. In some embodiments of the present invention the waste reservoir or a container housed within the waste reservoir may be disposable, so that a fresh, sterile waste reservoir or container, is introduced to the present invention for each analysis or at intervals between analyses. A skilled reader will recognize that other methods of disposing of the cells collected in the waste reservoir may also be incorporated in the system, device and method of the present invention, for example, such as means of removing the cells from the waste reservoir, a removable collection container incorporated in the waste reservoir in which cells may be collected and disposed of when the collection container is removed from the waste reservoir, cleansing means applied to the waste reservoir, or any other means of removing the cells collected in the waste reservoir.

The optical imaging system of the present invention, such as that shown in FIG. 2, may be incorporated in the analysis chamber, whereby the optical imaging system and method of utilizing the optical imaging system, as described herein, occurs while the cells flows through the analysis chamber. A detection window, as shown in FIGS. 3a-3f may further be incorporated in the analysis chamber, whereby when the cells flow through the analysis chamber the cells will flow over or through the detection window.

The fluidic movement of the cells within the present invention, whereby the cells pass through the disposable cartridge of the optical imaging system, for example, such as a microfluidic chip, may be entirely driven by the capillary force. As described herein, the application of capillary force to drive the cells may eliminate the need for complex fluidic driving components such as vacuum pumps to be incorporated in the present invention. Therefore, the present invention may not include any complex fluidic driving components, such as vacuum pumps.

The cell sample may flow through the disposable cartridge, such as a microfluidic chip. The sample may be mixed with reagents, for example, such as slow-dried chemical reagents. The reagents may be coated on the surface of the microfludic chip.

In one embodiment of the present invention a passive microfluidic mixer may be used to prepare the sample for subsequent optical analysis. The passive microfluidic mixer may be positioned in the preparation chamber or the analysis chamber. As an example, the passive microfluidic mixer may perform steps to prepare the sample so as to cause tagging the cells of interest with fluorescently labelled antibodies. The passive mixing approach may eliminate the need for more cumbersome active devices to be included in the present invention. The present invention may therefore represent a system and device having reduced overall instrument complexity in comparison to known prior art systems.

An interrogation line or area may be incorporated in the present invention to examine and measure the excited signal, for example, such as an excited fluorescence signal. The excited signal may be created by an optical fiber/light guide of the optical imaging system acting upon the cell sample flowing through the optical imaging system. The guide may be coated with excitation filter.

The interrogation line or area may be defined by the size of the detector, such as a CMOS detector or a CCD detector. Since the cell enumeration may be completed dynamically in the present invention, the achievement of a well behaved cells flowing pattern is preferred for the present invention. To achieve the well behaved cells flowing pattern, the microfluidic channel of the narrow interrogation region may be designed in such a way that the flow is strictly laminar. As an example, the channel may be designed to specific dimensions, such as dimensions that achieve a depth of about 15 micron and a width of less than about 500 microns. The specific dimensions of the channel may be chosen to achieve improved acquired image quality and increased accuracy of the dynamic cell detection.

Possible configurations of an optical imaging system of the cell/particle detection and analysis platform of the present invention are shown in FIGS. 1 and 2. The optical imaging system may incorporate a single optical filter or an array of filters positioned in front of an optical detector. This configuration of elements may eliminate the need for any additional components to achieve fluorescent detection in multi-colour or multiplexed analysis as are incorporated in prior art systems, devices and methods of cell analysis. Therefore, the present invention may not incorporate any additional moving components to achieve fluorescent detection in multi-colour or multiplexed analysis.

The entrance of the cell sample, for example, such as a blood sample, in the analysis chamber may trigger the detector in the optical imaging system of the present invention to start capturing images. The detector may capture images of a portion of the analysis chamber that is a detection window, as shown in FIGS. 3a-3f. The detection window may be positioned over or upon a portion of the disposable cartridge, or over or upon the whole of the cartridge. The sample may flow under or over the detection window, depending on the configuration and positioning of the detection window within the analysis chamber.

As the fluidic sample moves into and fills up the analysis chamber, the detector may continue capturing optical images over time, at regular or irregular intervals. Once the entire analysis chamber is completely filled, the image capturing process may stop.

The present invention may apply an analysis method to the images for the purpose of analyzing the sample and producing analysis results. As part of the analysis method all of the images captured by the detector may be combined. The analysis method may involve image analysis of the acquired pictures whereby the number of fluorescent particles of interest flown by under the detection window may be calculated. The calculation may result in the generation of a final cell count.

The present invention may involve slow fluidic movement of the cell sample inside the cartridge of the optical imaging system and large volume of the sample to be analyzed. Therefore, a significant and possibly large amount of images may need to be recorded and processed as part of the analysis method of the present invention. The images may be stored to a storage means that is internal or external to the device of the present invention. The storage means may be linked or otherwise connected to the present invention by way of a wired or wireless connection. For example, one embodiment of the present invention may comprise a portable device, such as handheld device, that incorporates the platform of the present invention. In such an embodiment of the present invention images of the cell sample flowing over or under the detection window that are captured by the detector of the optical imaging system may be stored in a memory unit that may be internal to the device. In another embodiment of the present invention, the images may be stored in a memory unit that is external to the device. An external memory unit may be linked to the device by a wired or wireless connection.

The analysis method of the present invention may further incorporate a graphical processing unit operable to process and analyze one or more of the images captured by the detector of the optical imaging system of the present invention. In one embodiment of the present invention, once the analysis chamber is filled with the cell sample, the detector of the optical imaging system, that may be a CMOS detector or a CCD detector, may capture one or more static optical scattering images of the sample inside the analysis chamber. The detector may capture scattering images either after the detector of the optical imaging system has captured all of the images required for the analysis method of the present invention to count the cells in accordance with the method described herein, or the detector may capture scattering images intermittently between capturing images required for the analysis method of the present invention to count the cells. The scattering images captured by the detector may be provide static exposures that may be utilized by the present invention to study the finer details of the cells sample, for example, such as a blood sample. The study of finer details may provide additional information regarding the sample or particular cells in the sample, for example, such as cell/particle morphology, optical density of the carrier solution, etc. This information may be utilized in diagnosis and/or monitoring of diseases and assays in relation to the person from whom the sample is obtained. For example, the information may be utilized to undertake malaria and tuberculosis diagnostics.

The present invention may incorporate software or other computer program elements. One embodiment of the present invention may incorporate an image analysis program operable to undertake the image analysis of the acquired pictures whereby the number of fluorescent particles of interest flown by under the detection window may be calculated and/or the final cell count, as described herein. The image analysis program may utilize the images captured by the detector of the optical imaging system. The images, that may be fluorescence images, may be transferred directly to the image analysis program by the detector, or may be transferred from the detector to a storage means of unit, and may be accessed by the image analysis program from the storage unit. The image analysis program may be operable to access images stored in the storage unit through a wired connection, or wirelessly.

The image analysis program may incorporate an algorithm, such as a custom designed algorithm, that is operable to track the cells of interest shown in the images captured by the detector. For example, the captured images may show the entrance region of the detection window, so that the entrance region of the detection window is scanned in every captured image frame. Through an analysis of the captured images in the order that the images were captured, the image analysis program may be operable to track new events in the particles/cell sample as the sample flows over or under the detection window.

The image analysis program may be operable to detect intensity levels in the samples. For example, images of fluorescent cell samples may be analyzed to detect groups of pixels in the images that display pre-determined intensity levels, for example, such as intensity levels exceeding a pre-determined threshold that is set in the image analysis program. As an example, in an embodiment of the present invention that achieves strictly laminar fluidic flow of the cell sample inside the microchannels, the image analysis program may identify small regions where a fluorescent particle is expected in subsequent frames. Due to the fact that only a small portion of the entirety of each image may be processed by the image analysis program in this analysis method the processing speed may be increased and the computing power requirements may be reduced. These outcomes may occur in particular if the frame rate is above about 15 frames per second.

For the imaging analysis program to track the location of fluorescently labelled subjects within each image frame, a virtual bounding box may be placed on each cell, whereby the maximum and minimum x and y coordinates of each cell may be identified and recorded by the imaging analysis program. The center position of each bounding box may be computed and marked as the cell's current position. This process may be repeated for each image frame captured and analyzed to produce a final enumeration result.

In addition to the entrance scanning of target population by the imaging analysis program, cells of interest may also be detected and counted upon the cell exiting the detection window area. The imaging analysis program may undertake this type of exiting cell analysis to ensure the accuracy of the counting of the cells upon entering the detection window area. This exiting cell analysis can therefore provide verification of the count of the cells entering the detection window area. Therefore, incorporating the exiting cell analysis in the analysis method of the present invention performed by the imaging analysis program may diminish, virtually eliminate or eliminate errors introduced by cells remaining attached to the surfaces of microchannels inside the detection window. This in turn may increase the accuracy of the results of the present invention.

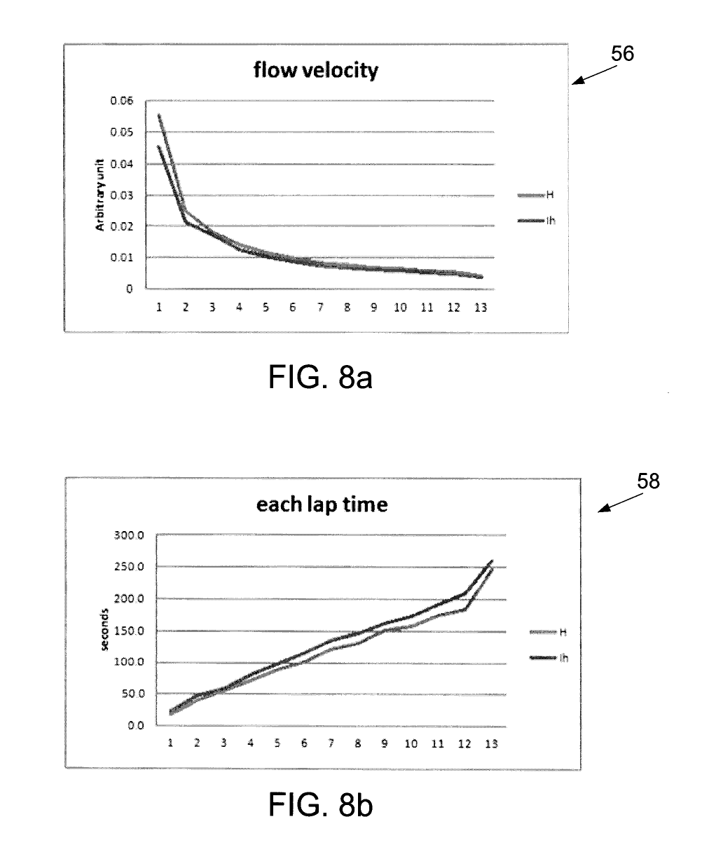

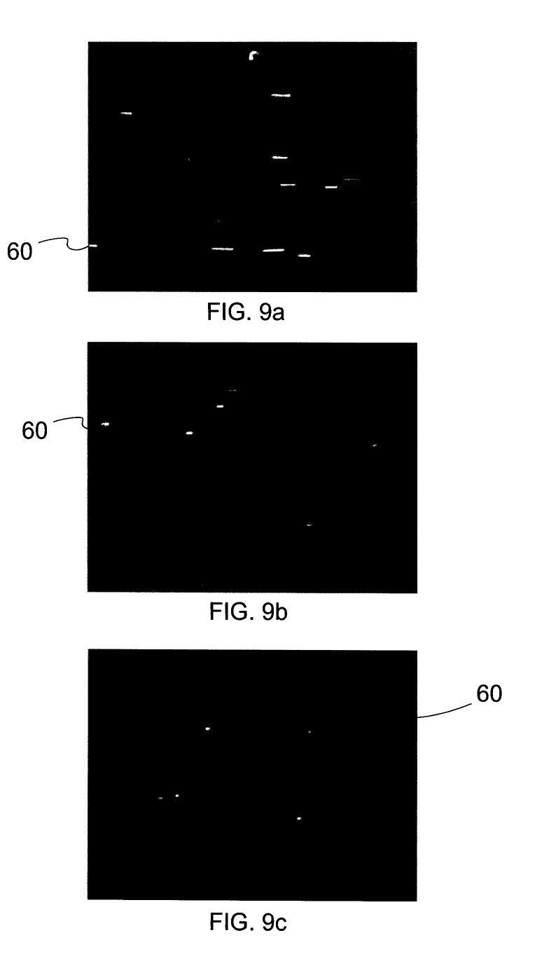

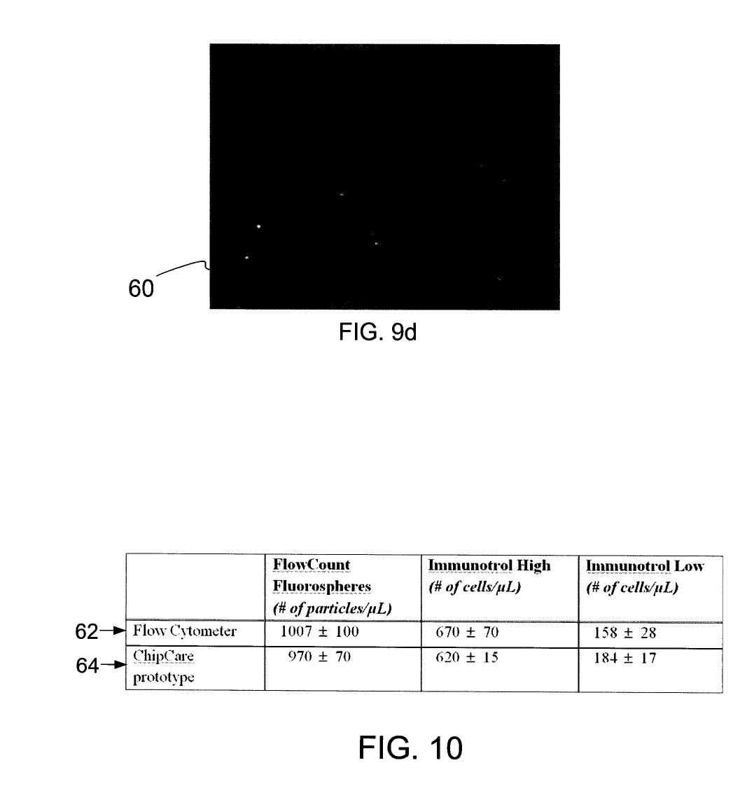

Aspects of flow speed characterization may be utilized to design the microfluidic devices or chips. For example, in one embodiment of the present invention microfluidic chips were designed based on numerical simulation results obtained. The fluid velocity may be estimated from such collected data, such as the data collected from the images shown in FIGS. 9a-9d, and may be plotted, as shown in FIGS. 8a and 8b. It may be that experimental results agree well with the theoretical expectations. The plotting may show flow velocity 56 representing the fluid flow speed, as shown in FIG. 8a, or the plotting may show each lap time 58 representing the channel filling time, as shown in FIG. 8b. The plotting shown in FIGS. 8a and 8b correspond to the serpentine microfluidic chip structure shown in FIGS. 6a and 6b. Thus, a numerical model may be created before the microfluidic chip design occurs. The model in this example simulated fluidic motion in a simple capillary microfluidic system with a straight channel connecting two reservoirs.

The present invention may incorporate a means of performing optimization of parameters applied by the present invention. In an embodiment of the present invention that undertakes dynamic counting to determine the cell/particle statistics, the exposure time of the optical detector may be the most important parameter in optimizing the counting accuracy. The performance of the present invention may be maximized when the signal to background noise ratio is increased. The ability to achieve this maximization is affected by the exposure time of the images captured by the detection of the optical imaging system. If the integration time of the detector is too short, the fluorescence signal captured by the detector may be low and may compromise the signal to background ratio. On the other hand, if the integration time is too long, the cells in the sample may be travelling too fast for the detector to capture an image of such cells. If cells flow by and are not captured in an image by the detector, and therefore are not registered, this may lead to counting errors and errors in the analysis results. The optimal integration time of the present invention should allow the fluorescently labeled cells in the sample to produce sufficient signal, compared with the background noise. The electronic circuitry that drives the detector should also be fast enough to capture all the cells/particles of interest with a proper sampling rate.

An example of the effect of the detector exposure time is shown in FIGS. 9a-9d. FIG. 9a shows an image captured by the detector of the optical imaging system at a 50 ms exposure, S/B: 3/2. FIG. 9b shows an image captured by the detector of the optical imaging system at a 25 ms exposure, S/B: 1300/900. FIG. 9c shows an image captured by the detector of the optical imaging system at a 15 ms exposure, S/B: 750/550. FIG. 9d shows an image captured by the detector of the optical imaging system at a 10 ms exposure, S/B: 695/500. A comparison of FIGS. 9a-9d shows that the cells shape 60 (shown as light shapes within the dark background in the images) appears more circular at shorter exposure, as the signal to background ratio is lower at a shorter exposure.

For purposes of showing the benefits of the present invention over the prior art a comparison was made of an embodiment of the present invention to the gold standard flow cytometer. Using this approach, enumeration measurements were conducted on 6-um polymer microspheres, conjugated with Phycoerythrin (PE) dyes, in phosphate buffered saline (PBS) solutions. The initial experiments were performed on an Olympus BX50 upright fluorescence microscope. Band pass filter sets were used for fluorescence excitation and emission measurements. An average count of 1007 particles/μL was obtained, while the flow cytometer produced a count of 970 particles/μL using the same sample.

The embodiment of the present invention utilized for the comparison was comprised of off-the-shelf optical and mechanical components. The comparison involved the measurement of CD4 T cell concentration in a whole blood sample. The microfluidic chips were tested using immonotrols, which were stabilized blood samples used to calibrate flow cytometer systems, at both high and low concentrations. The same fluorescent dyes were used in testing the stabilized blood sample. The testing produced an average count of 620 cells per microliter whereas the flow cytometer measured 670 cells per microliter.

A comparison of the results of the tests on the gold standard flow cytometer (prior art) 62 and the present invention described as a ChipCare prototype 64 are show in FIG. 10, wherein the wide field dynamic counting system and flow cytometer test result are shown.

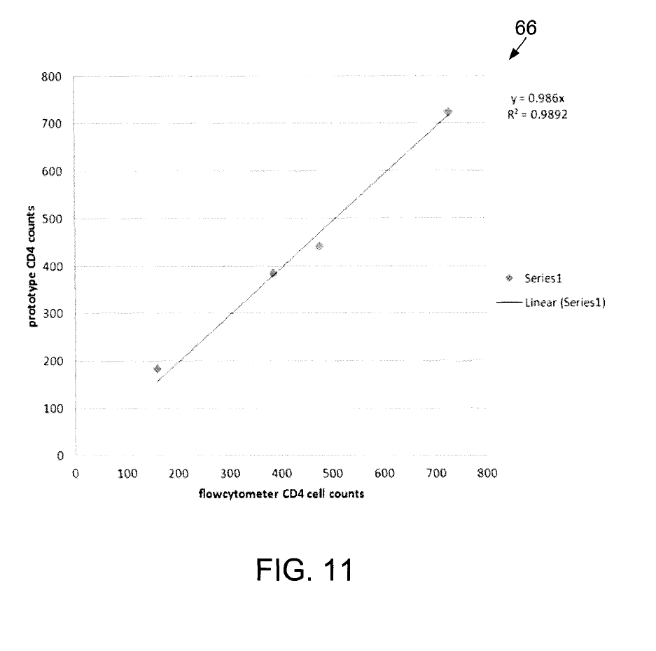

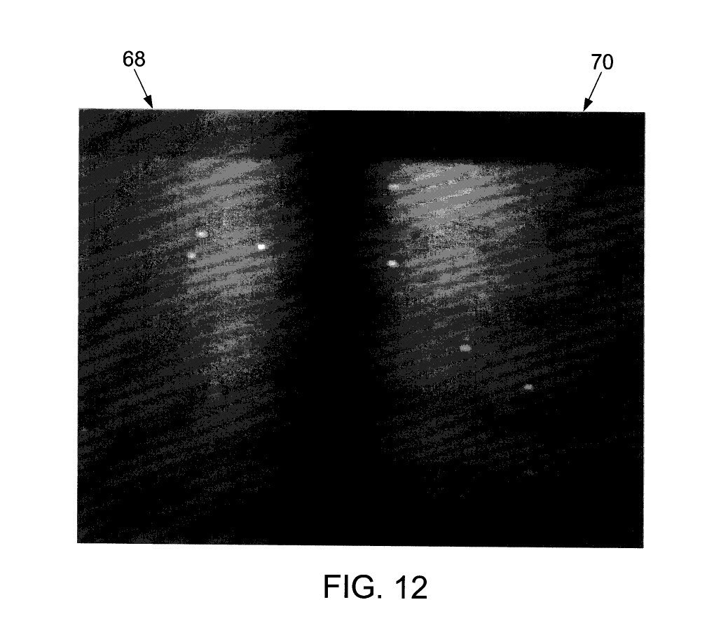

A linearity test was also performed to show the differences between the prior art flow cytometer and the present invention. Results of the test are shown in the table 66 of FIG. 11. A range of cell populations of interest to CD4 enumeration assay was tested on the platform of the present invention, from 150 per μL to 720 per μL, as shown in FIG. 11. The measurements were done in direct comparison to the conventional, prior art clinical flow cytometers, and were conducted over a range of cell populations of clinical relevance. The results of the comparison yielded a 98.6% agreement between two measurements. (Each data point shown in the table 66 of FIG. 11 is the average of the counting results obtained for that range.)

The present invention may be operable to perform single colour or multiple colour imaging. As shown in FIG. 12, an embodiment of the present invention may be operable to perform two colour imaging. Through multiple colour imaging the present invention exhibits multiplexing capabilities, and thereby may generate statistics of an individual cell group population and differential statistics of multiple cell types. Filter sets may be incorporated in the optical imaging system of the present invention that include two half-moon shaped optical filters combined and placed in front of the optical detector. The two half-moon shaped optical filters may be placed together side-by-side in an embodiment of the present invention. The two half-moon shaped optical filters may further be custom designed for embodiments of the present invention.

Particles labelled with specific fluorochrome may be present in the corresponding panel/region of the optical detector. In a demonstration example of a two colour imaging of an embodiment of the present invention, as shown in FIG. 12, two fluorescent dyes used were PE and PE-Cy5. Each dye was conjugated to a specific cell, so that CD4 cells were tagged with PE dye while CD45 cells were labelled with PE-Cy5 molecules. The left half 68 of the detection region captured the image of the sample at the emission wavelength of the first fluorescent label (PE), appearing as a green colour, while the right panel 70 of the detection region recorded images of the sample at the emission wavelength of the second fluorescent label (PE-Cy5), appearing as a red colour. Two cell populations were measured using this approach in the demonstration, CD4 and CD45. Through a combination and comparison of these two sets of images populations of labelled cells can be measured by the present invention.

It will be appreciated by those skilled in the art that other variations of the embodiments described herein may also be practiced without departing from the scope of the invention. Other modifications are therefore possible.

1. A cell detection and analysis system characterized in that it comprises:
(a) a fluidic chip incorporating a microfluidic channel operable for one or more cells to flow within the microfludic channel;
(b) an optical source positioned to be directed to the fluidic chip or a portion of the fluidic chip; and
(c) a detection module operable to capture one or more images of the one or more tells flowing within the fluidic chip, and operable to establish multi-colour fluorescence detection assays while the fluidic chip, optical source and detection module remain stationary within the system, said multi-colour fluorescence detection assays being multi-wavelength detection assays of the fluidic chip incorporating detection points that are positioned to be any of the following: serial; parallel; or a combination of serial and parallel.

2. The system of claim 1, characterized in that the fluidic chip incorporates a detection window and the detection module is operable to capture images of the one or more cells flowing within the fluidic, chip through the detection window in continuous manner.

3. The system of claim 1, characterized in that the optical source is a light source that is positioned either above or below the fluidic chip.

4. The system of claim 1, characterized in that the detection module incorporates a CMOS detector or a CCD detector.

5. The system of claim 1, characterized in that the detection module incorporates an image analysis program operable to analyze the one or more images captured by the detection module to produce analysis results.

6. The system of claim 5, characterized in that the image analysis program produces diagnostic results.

7. The system of claim 1, characterized in that the system is portable.

8. The system of claim 1, characterized in that the fluidic chip, optical source and detection module may be incorporated within a single housing.

9. A method for cell detection analysis, characterized in that comprises the following steps:
(a) introducing a cell sample of one or more cells to a fluidic chip;
(b) flowing the cell sample through a microfluidic channel within the fluidic chip; and
(c) operating an optical imaging module to analyze the cell sample flowing within the fluidic chip, and to establish multi-colour fluorescence detection assays while the fluidic chip and the optical imaging module remain stationary.

10. The method of claim 9, characterized in that it comprises the further steps of operating the optical imaging module to analyze the cell sample flowing within the fluidic chip by said optical imaging module:
(i) operating a detector to capture one or more images of the cell sample flowing past a detection window section of the fluidic chip;
(ii) operating an image analysis program to analyze the one or more images captured by the detector, and said image analysis program generating cell analysis results relating to the cell sample.

11. The method of claim 10, characterized in that it comprises the further step of the image analysis program generating diagnostic results relating to the cell sample.

12. The method of claim 9, characterized in that it comprises the further step of the optical imaging module applying one or more calculations and one or more algorithms to analyze the cell sample.

13. The method of claim 9, characterized in that it comprises the further steps of:
(i) creating a portable device that incorporates the fluidic chip and optical imaging module;
(ii) a user carrying the portable device to various locations to perform cell detection and analysis.

14. The method of claim 13, characterized in that it comprises the further step of carrying the portable device to perform cell detection and analysis in one or more of the following: one or more remote locations; one or more developing locations; one or more developed locations.

15. The method of claim 9, characterized in that it comprises the further step of storing the analysis of the cell sample of the optical imaging system in a storage means.

16. An apparatus for cell detection and analysis characterized in that it comprises:
(a) one or more housings;
(b) a fluidic chip incorporating a microfluidic channel that one or more cells of a cell sample flows through within the fluidic chip;
an optical imaging system incorporated in one of the one or more housings, said optical imaging system being operable to capture one or more images of the one or more cells flowing within the fluidic chip when the optical imaging system is positioned to be directed to the fluidic chip or a portion of the fluidic chip, and further being operable to establish multi-colour fluorescence detection assays while the fluidic chip and the optical imaging system remain stationary within the apparatus; and
(d) a cell sample analysis means operable to utilize the one of more images to generate a cell sample analysis result.

17. The apparatus of claim 16, characterized in that it further comprises the fluidic chip having:
(i) an inlet through which the cell sample is introduced to the microfluidic channel;
(ii) an outlet through which the cell sample may be removed from the fluidic chip; and
(iii) posts positioned within the fluidic chip.

18. The apparatus of claim 17, characterized in that it further comprises a waste reservoir positioned near the outlet, said waste reservoir being operable to collect the cell sample after the cell sample has flowed through the microfluidic chip.

19. The apparatus of claim 16, characterized in that it further comprises the fluidic chip, the optical imaging system and the cell sample analysis means being incorporated in the one housing that is a portable, handheld housing.

20. The apparatus of claim 16, characterized in that it further comprises a handheld device that is connected to the apparatus whereby the cell sample analysis may be presented to a user by the handheld device.

21. The apparatus of claim 16, characterized in that it further comprises the optical imaging system and cell sample analysis means being operable to apply multi-fluorescence detection.

22. A apparatus for cell detection and analysis characterized in that it comprises;
(a) one housing that is a portable, handheld housing;
(b) a fluidic chip incorporating a microfluidic channel that one or more cells of a cell sample flows through within the fluidic chip;
(c) an optical imaging system incorporated in one of the one housing, said optical imaging system being operable to capture one or more images of the one or more cells flowing within the fluidic chip when the optical imaging system is positioned to be directed to the fluidic chip or a portion of the fluidic chip, and further being operable to establish multi-colour fluorescence detection assays while the fluidic chip and the optical imaging system remain stationary within the apparatus; and
(d) a cell sample analysis means operable to utilize the one or more image to generate a cell sample analysis result.
